(19) United States
(12) Patent Application Publication
(10) Pub. No.: US 2023/0248735 A1
(43) Pub. Date: Aug. 10, 2023

(54) COMPOSITIONS FOR PREVENTING OR TREATING CHRONIC OBSTRUCTIVE PULMONARY DISEASES (COPD)

(71) Applicant: Chong Kun Dang Pharmaceutical Corp., Seoul (KR)

(72) Inventors: Young II Choi, Gyeonggi-do (KR); Nina Ha, Gyeonggi-do (KR); Dong Hyeon Suh, Gyeonggi-do (KR)

(21) Appl. No.: 17/768,571

(22) PCT Filed: Oct. 22, 2020
(86) PCT No.: PCT/IB2020/059919

§ 371 Date: Apr. 13, 2022

(30) Foreign Application Priority Data
Oct. 23, 2019 (KR) 10-2019-0132501

(51) Int. Cl. A61K 31/5377; A61P 11/00; A61K 31/496; A61K 31/5375; A61K 31/495; A61K 31/454; A61K 31/506; A61K 31/4545; A61K 31/444; A61K 31/4439; A61K 31/551; A61K 31/4427; A61K 31/4453; A61K 31/4525; A61K 31/397; A61K 31/4025; A61K 31/451; A61K 31/445; A61K 31/40; A61K 31/453; A61K 31/553

(52) U.S. Cl. CPC A61K 31/5377; A61K 31/40; A61K 31/397; A61K 31/444; A61K 31/445; A61K 31/451; A61K 31/453; A61K 31/454; A61K 31/495; A61K 31/496; A61K 31/506; A61K 31/551; A61K 31/553; A61K 31/4025; A61K 31/4427; A61K 31/4439; A61K 31/4453; A61K 31/4525; A61K 31/4545; A61K 31/5375; A61P 11/00

(57) ABSTRACT

The present invention relates to a pharmaceutical composition for preventing or treating chronic obstructive pulmonary disease, containing a compound represented by a chemical formula I, optical isomers thereof or pharmaceutically acceptable salts thereof as an effective ingredient, a method for preventing or treating chronic obstructive pulmonary disease by using the compound or the composition, and a use of the compound or the composition in preparation of a medicament for treating chronic obstructive pulmonary disease.

### SEQUENCE LISTING

This application contains a Sequence Listing that has been submitted electronically as an ASCII text file named 45695-0020US1.txt. The ASCII text file, created on Nov. 10, 2022, is 1,917 bytes in size. The material in the ASCII text file is hereby incorporated by reference in its entirety.

### TECHNICAL FIELD

The present invention relates to a pharmaceutical composition for preventing or treating chronic obstructive pulmonary disease, comprising a compound represented by a chemical formula I, optical isomers thereof or pharmaceutically acceptable salts thereof as an effective ingredient, a method for preventing or treating chronic obstructive pulmonary disease by using the compound or the composition, and a use of the compound or the composition in preparation of a medicament for treating chronic obstructive pulmonary disease.

### BACKGROUND OF THE INVENTION

Chronic obstructive pulmonary disease (COPD) is a disease which results from pathological changes in the bronchioles and the lung parenchyma caused by airway and lung parenchymal inflammation, and has a feature of having obstructive bronchiolitis and pulmonary emphysema (pulmonary parenchymal destruction). Types of chronic obstructive pulmonary disease include chronic obstructive bronchitis, chronic bronchiolitis, emphysema and the like.

Smoking is thought to be the most important cause of COPD. Smoking acts as a strong toxic substance in the lung tissues to promote the production of oxides, pro-inflammatory factors, and chemotactic factors, which promotes excessive migration of inflammatory cells such as neutrophils. Inflammatory cells that have migrated into the lung tissues secrete many inflammatory mediators, further exacerbating the inflammation in the lung tissues. As a mediator for promoting such an inflammatory response, TNF-α, etc., are mainly known and used as an important marker in the inflammatory response caused by tobacco smoke.

In a recent study, it has been reported that an epithelial-to-mesenchymal transition (EMT) may be one of the pathogenic mechanisms of chronic obstructive disease (Courtney JM, et al. Cells Tissues Organs. 2017;203(2):99-104), and it has been also reported that FN-EDA (extra domain-A-containing fibronectin), one of the major proteins in the extracellular matrix (ECM), is found in the airways of COPD patients (Annoni R, et al. Eur Respir J. 2012;40(6): 1362-73).

Currently, the substances used in the treatment of chronic obstructive pulmonary disease have been mainly developed to ameliorate inflammation in the lung tissues, and steroids, anti-inflammatory drugs, etc. have been mainly used. However, these substances cause various side effects such as immunosuppression, tolerance and the like, and thus are unsuitable for patients with chronic obstructive pulmonary disease who require a long-term treatment. Accordingly, there is an urgent need for developing a drug capable of effectively treating COPD.

[Related Art Reference] (Patent Document 1) Korean Patent Publication No. 10-2014-0128886

### DETAILED DESCRIPTION OF THE INVENTION

### Technical Problem

The present invention provides a pharmaceutical composition for preventing or treating chronic obstructive pulmonary disease, comprising a compound represented by a chemical formula I, optical isomers thereof or pharmaceutically acceptable salts thereof as an effective ingredient.

The present invention provides a method for preventing or treating chronic obstructive pulmonary disease, comprising administering said pharmaceutical composition into an individual.

The present invention provides a use of said composition in preparation of a medicament for preventing or treating chronic obstructive pulmonary disease.

The present invention provides a method for preventing or treating chronic obstructive pulmonary disease, including administering a therapeutically effective amount of a compound represented by the chemical formula I, optical isomers thereof or pharmaceutically acceptable salts thereof into an individual.

The present invention provides a use of a compound represented by the chemical formula I, optical isomers thereof or pharmaceutically acceptable salts thereof for preventing or treating chronic obstructive pulmonary disease.

The present invention provides a use of a compound represented by the chemical formula I, optical isomers thereof or pharmaceutically acceptable salts thereof in preparation of a medicament for treating chronic obstructive pulmonary disease.

### Technical Solution

Under the above-described background, the present inventors have made research efforts to develop a therapeutic agent for chronic obstructive pulmonary disease, and thus have confirmed that a compound according to the present invention shows an inhibitory effect on the infiltration of immune cells, the infiltration of inflammatory cells, and the expression of inflammatory cytokines in lung tissues in a mouse with induced COPD, as well as an inhibitory effect on TGF-β1-induced EMT and the expression of FN-EDA, which is a major ECM protein, thereby completing the present invention.

This is described in detail as follows. Meanwhile, each description and embodiment disclosed in the present invention may be also applied to other descriptions and embodiments thereof, respectively. In other words, all the combinations of various elements disclosed in the present invention fall within the scope of the present invention. Also, it cannot be seen that the scope of the present invention is limited to the specific description described below.

The present invention provides a pharmaceutical composition for preventing or treating chronic obstructive pulmonary disease, comprising a compound represented by a following chemical formula I, optical isomers thereof or pharmaceutically acceptable salts thereof as an effective ingredient:

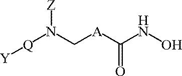

wherein
Ais

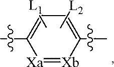

Xa and Xb are each independently CH or N,
L₁ and L₂ are each independently hydrogen, halogen, —CF₃, or -C₁₋₃ straight or branched chain alkyl,
Q is C(=O), S(=O)₂, S(=O) or C(=NH),
Y is selected from a following group:

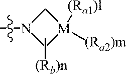

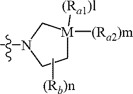

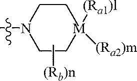

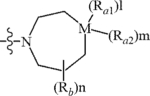

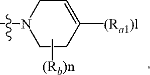

M is C, N, O, S or S(=O)₂ (at this time, if M is C, 1 and m are 1; if M is N, 1 is 1 and m is 0; and if M is O, S or S(=O)₂, 1 and m are 0),
Rₐ₁ and Rₐ₂ are each independently hydrogen; hydroxy; -C₁₋₄ straight or branched chain alkyl, which is unsubstituted or substituted with at least one halogen; -C₁₋₄ straight or branched chain alcohol; benzhydryl; -C₁₋₄ straight or branched chain alkyl, which is substituted with a saturated or unsaturated five to seven-membered heterocyclic compound having one to three heteroatoms out of N, O or S as a ring member (at this time, the heterocyclic compound may be unsubstituted or at least one hydrogen may be optionally substituted with OH, OCH₃, CH₃, CH₂CH₃ or halogen); a saturated or unsaturated five to seven-membered heterocyclic compound having one to three heteroatoms out of N, O or S as a ring member (at this time, the heterocyclic compound may be unsubstituted or at least one hydrogen may be optionally substituted with OH, OCH₃, CH₃, CH₂CH₃ or halogen); phenyl, which is unsubstituted or in which at least one hydrogen is substituted with halogen, C₁₋₄ alkoxy, C₁₋₂ alkyl or hydroxy; benzyl, which is unsubstituted or in which at least one hydrogen is substituted with halogen, C₁₋₄ alkoxy, C₁₋₂ alkyl or hydroxy; —S(═O)₂CH₃; halogen; -C₁₋₆ straight or branched chain alkoxy; -C₂₋₆ alkoxyalkyl; -C(=O)Rₓ, in which Rₓ is C₁₋₃ straight or branched chain alkyl or C₃₋₁₀ cycloalkyl;

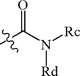

, in which R_{c} and R_{d} are independently hydrogen or C₁₋₃ straight or branched chain alkyl;

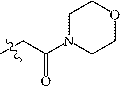

or

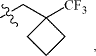

n is an integer of 0, 1 or 2,
R_{b} is hydrogen; hydroxy; -C₁₋₆ straight or branched chain alkyl, which is unsubstituted or in which at least one hydrogen is substituted with halogen; —C(═O)CH₃; -C₁₋₄ straight or branched chain hydroxyalkyl; -C₁₋₆ straight or branched chain alkoxy; -C₂₋₆ straight or branched chain alkoxyalkyl; —CF₃; halogen; or

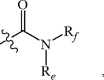

Rₑ and R_{f} are each independently hydrogen or -C₁₋₃ straight or branched chain alkyl, and
Z is selected from a following group:

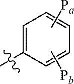

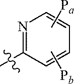

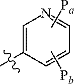

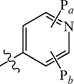

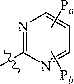

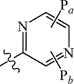

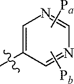

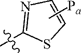

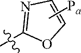

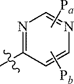

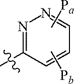

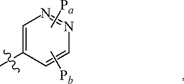

Pₐ and P_{b} are each independently

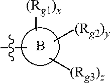

; hydrogen; hydroxy; -C₁₋₄ straight or branched chain alkyl, which is unsubstituted or in which at least one hydrogen is substituted with halogen; halogen; —CF₃; —OCF₃; —CN ; -C₁₋₆ straight or branched chain alkoxy; -C₂₋₆ straight or branched chain alkyl alkoxy; —CH₂F; or -C₁₋₃ alcohol,
in which

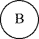

is a ring selected from phenyl, pyridine, pyrimidine, thiazole, indole, indazole, piperazine, quinoline, furan, tetrahydropyridine, piperidine or a following group:

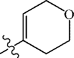

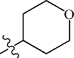

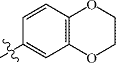

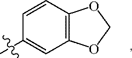

x, y and z are each independently an integer of 0 or 1, and
R_{g1}, R_{g2} and R_{g3} are each independently selected from hydrogen; hydroxy; -C₁₋₃ alkyl; —CF₃; -C₁₋₆ straight or branched chain alkoxy; -C₂₋₆ straight or branched chain alkyl alkoxy; —C(═O)CH₃; -C₁₋₄ straight or branched chain hydroxyalkyl; —N(CH₃)₂; halogen; phenyl; —S((═O)₂)CH₃; or a following group:

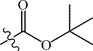

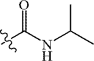

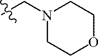

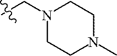

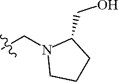

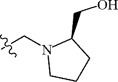

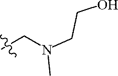

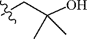

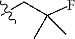

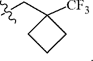

In the present invention, the compound represented by the above chemical formula I may be a compound represented by a following chemical formula Ia:

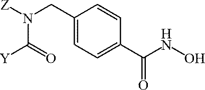

wherein
Y is selected from a following group:

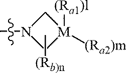

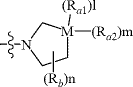

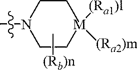

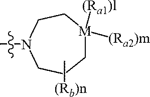

in which M, 1, m, n, Rₐ₁, Rₐ₂, and R_{b} are each the same as defined in the above chemical formula I,
Z is

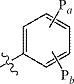

, and
Pₐ and P_{b} are each independently hydrogen; hydroxy; -C₁₋₄ straight or branched chain alkyl, which is unsubstituted or in which at least one hydrogen is substituted with halogen; halogen; —CF₃; —OCF₃; —CN; -C₁₋₆ straight or branched chain alkoxy; -C₂₋₆ straight or branched chain alkyl alkoxy; —CH₂F; or-C₁₋₃ alcohol.

According to one embodiment aspect of the present invention,
Y is selected from a following group:

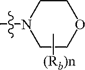

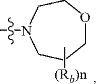

in which n and R_{b} are each the same as defined in the above chemical formula I,
Z is

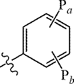

, and
Pₐ and P_{b} are each independently hydrogen; halogen; —CF₃; or -C₁₋₆ straight or branched chain alkoxy.

In the present invention, “halogen” is F, Cl, Br or I.

According to a specific embodiment of the present invention, the compound represented by the above chemical formula I may be a compound described in a following table.

| Compound | Structure | Compound | Structure |
|---|---|---|---|
| 252 | 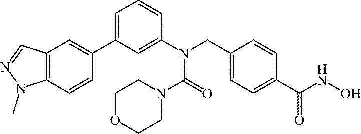 [SMILES: Cn1ncc2cc(-c3cccc(N(Cc4ccc(C(=O)NO)cc4)C(=O)N4CCOCC4)c3)ccc21] | 262 | 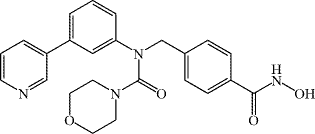 [SMILES: O=C(NO)c1ccc(CN(C(=O)N2CCOCC2)c2cccc(-c3cccnc3)c2)cc1] |
| 253 | 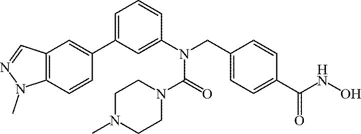 [SMILES: CN1CCN(C(=O)N(Cc2ccc(C(=O)NO)cc2)c2cccc(-c3ccc4c(cnn4C)c3)c2)CC1] | 263 | 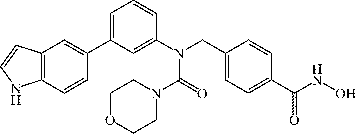 [SMILES: O=C(NO)c1ccc(CN(C(=O)N2CCOCC2)c2cccc(-c3ccc4[nH]ccc4c3)c2)cc1] |
| 254 | 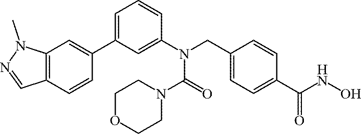 [SMILES: Cn1ncc2ccc(-c3cccc(N(Cc4ccc(C(=O)NO)cc4)C(=O)N4CCOCC4)c3)cc21] | 279 | 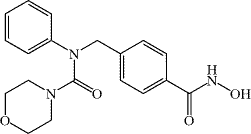 [SMILES: O=C(NO)c1ccc(CN(C(=O)N2CCOCC2)c2ccccc2)cc1] |
| 255 | 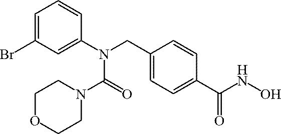 [SMILES: O=C(NO)c1ccc(CN(C(=O)N2CCOCC2)c2cccc(Br)c2)cc1] | 280 | 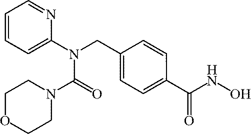 [SMILES: O=C(NO)c1ccc(CN(C(=O)N2CCOCC2)c2ccccn2)cc1] |
| 256 | 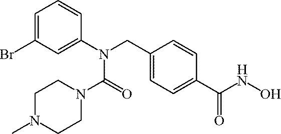 [SMILES: CN1CCN(C(=O)N(Cc2ccc(C(=O)NO)cc2)c2cccc(Br)c2)CC1] | 281 | 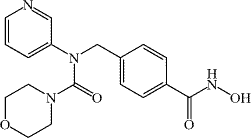 [SMILES: O=C(NO)c1ccc(CN(C(=O)N2CCOCC2)c2cccnc2)cc1] |
| 260 | 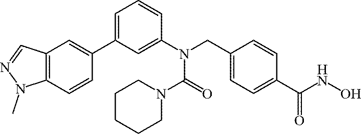 [SMILES: Cn1ncc2cc(-c3cccc(N(Cc4ccc(C(=O)NO)cc4)C(=O)N4CCCCC4)c3)ccc21] | 309 | 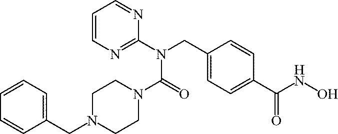 [SMILES: O=C(NO)c1ccc(CN(C(=O)N2CCN(Cc3ccccc3)CC2)c2ncccn2)cc1] |
| 261 | 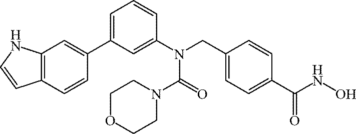 [SMILES: O=C(NO)c1ccc(CN(C(=O)N2CCOCC2)c2cccc(-c3ccc4cc[nH]c4c3)c2)cc1] | 311 | 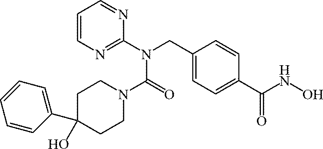 [SMILES: O=C(NO)c1ccc(CN(C(=O)N2CCC(O)(c3ccccc3)CC2)c2ncccn2)cc1] |
| 312 | 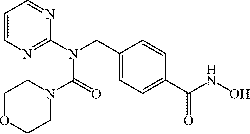 [SMILES: O=C(NO)c1ccc(CN(C(=O)N2CCOCC2)c2ncccn2)cc1] | 334 | 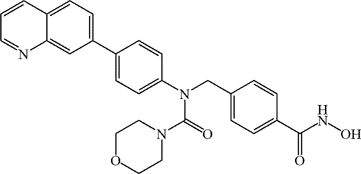 [SMILES: O=C(NO)c1ccc(CN(C(=O)N2CCOCC2)c2ccc(-c3ccc4cccnc4c3)cc2)cc1] |
| 313 | 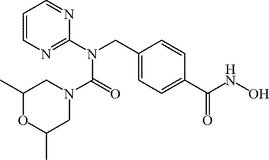 [SMILES: CC1CN(C(=O)N(Cc2ccc(C(=O)NO)cc2)c2ncccn2)CC(C)O1] | 335 | 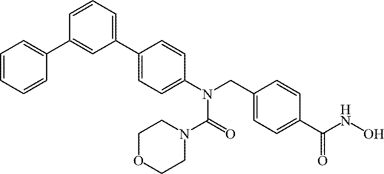 [SMILES: O=C(NO)c1ccc(CN(C(=O)N2CCOCC2)c2ccc(-c3cccc(-c4ccccc4)c3)cc2)cc1] |
| 329 | 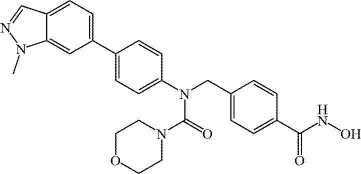 [SMILES: Cn1ncc2ccc(-c3ccc(N(Cc4ccc(C(=O)NO)cc4)C(=O)N4CCOCC4)cc3)cc21] | 336 | 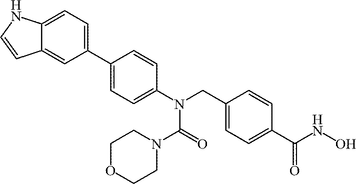 [SMILES: O=C(NO)c1ccc(CN(C(=O)N2CCOCC2)c2ccc(-c3ccc4[nH]ccc4c3)cc2)cc1] |
| 330 | 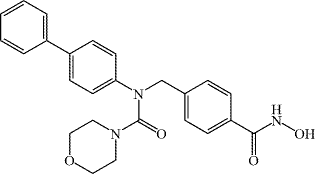 [SMILES: O=C(NO)c1ccc(CN(C(=O)N2CCOCC2)c2ccc(-c3ccccc3)cc2)cc1] | 337 | 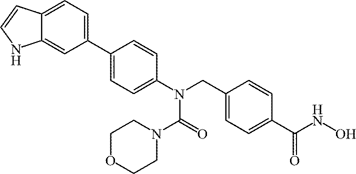 [SMILES: O=C(NO)c1ccc(CN(C(=O)N2CCOCC2)c2ccc(-c3ccc4cc[nH]c4c3)cc2)cc1] |
| 331 | 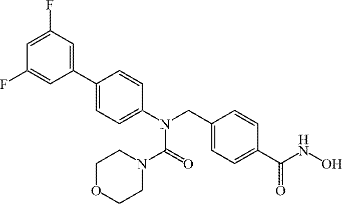 [SMILES: O=C(NO)c1ccc(CN(C(=O)N2CCOCC2)c2ccc(-c3cc(F)cc(F)c3)cc2)cc1] | 338 | 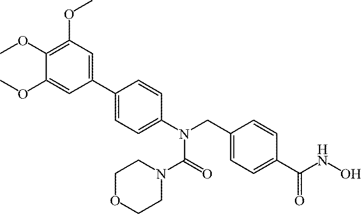 [SMILES: COc1cc(-c2ccc(N(Cc3ccc(C(=O)NO)cc3)C(=O)N3CCOCC3)cc2)cc(OC)c1OC] |
| 332 | 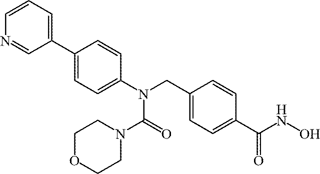 [SMILES: O=C(NO)c1ccc(CN(C(=O)N2CCOCC2)c2ccc(-c3cccnc3)cc2)cc1] | 339 | 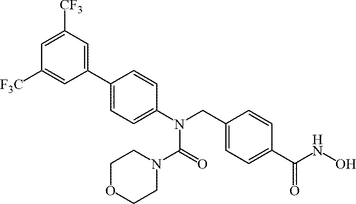 [SMILES: O=C(NO)c1ccc(CN(C(=O)N2CCOCC2)c2ccc(-c3cc(C(F)(F)F)cc(C(F)(F)F)c3)cc2)cc1] |
| 333 | 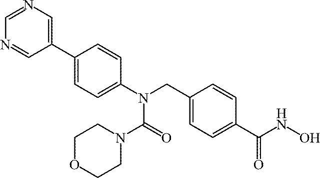 [SMILES: O=C(NO)c1ccc(CN(C(=O)N2CCOCC2)c2ccc(-c3cncnc3)cc2)cc1] | 340 | 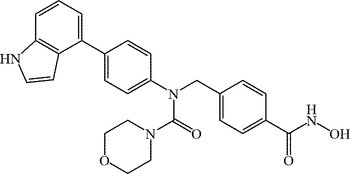 [SMILES: O=C(NO)c1ccc(CN(C(=O)N2CCOCC2)c2ccc(-c3cccc4[nH]ccc34)cc2)cc1] |
| 341 | 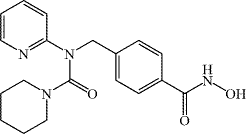 [SMILES: O=C(NO)c1ccc(CN(C(=O)N2CCCCC2)c2ccccn2)cc1] | 356 | 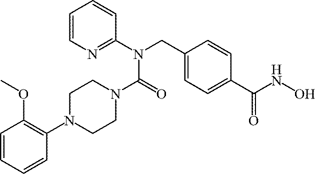 [SMILES: COc1ccccc1N1CCN(C(=O)N(Cc2ccc(C(=O)NO)cc2)c2ccccn2)CC1] |
| 342 | 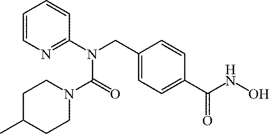 [SMILES: CC1CCN(C(=O)N(Cc2ccc(C(=O)NO)cc2)c2ccccn2)CC1] | 357 | 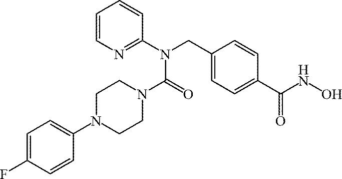 [SMILES: O=C(NO)c1ccc(CN(C(=O)N2CCN(c3ccc(F)cc3)CC2)c2ccccn2)cc1] |
| 343 | 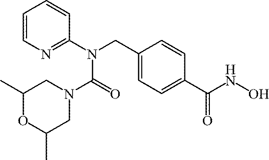 [SMILES: CC1CN(C(=O)N(Cc2ccc(C(=O)NO)cc2)c2ccccn2)CC(C)O1] | 358 | 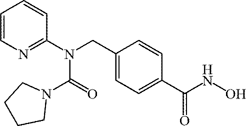 [SMILES: O=C(NO)c1ccc(CN(C(=O)N2CCCC2)c2ccccn2)cc1] |
| 352 | 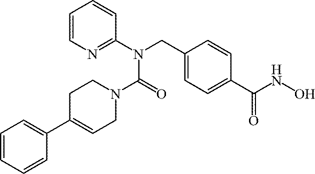 [SMILES: O=C(NO)c1ccc(CN(C(=O)N2CC=C(c3ccccc3)CC2)c2ccccn2)cc1] | 370 | 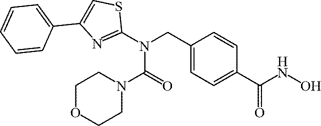 [SMILES: O=C(NO)c1ccc(CN(C(=O)N2CCOCC2)c2nc(-c3ccccc3)cs2)cc1] |
| 353 | 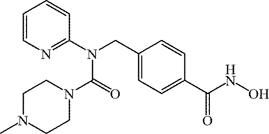 [SMILES: CN1CCN(C(=O)N(Cc2ccc(C(=O)NO)cc2)c2ccccn2)CC1] | 371 | 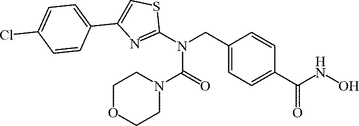 [SMILES: O=C(NO)c1ccc(CN(C(=O)N2CCOCC2)c2nc(-c3ccc(Cl)cc3)cs2)cc1] |
| 354 | 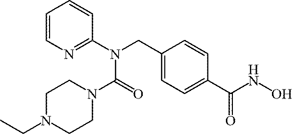 [SMILES: CCN1CCN(C(=O)N(Cc2ccc(C(=O)NO)cc2)c2ccccn2)CC1] | 372 | 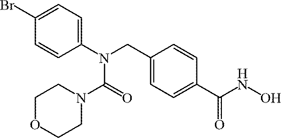 [SMILES: O=C(NO)c1ccc(CN(C(=O)N2CCOCC2)c2ccc(Br)cc2)cc1] |
| 355 | 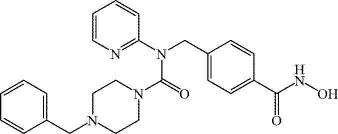 [SMILES: O=C(NO)c1ccc(CN(C(=O)N2CCN(Cc3ccccc3)CC2)c2ccccn2)cc1] | 374 | 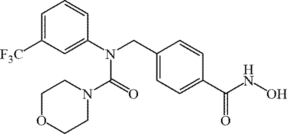 [SMILES: O=C(NO)c1ccc(CN(C(=O)N2CCOCC2)c2cccc(C(F)(F)F)c2)cc1] |
| 376 | 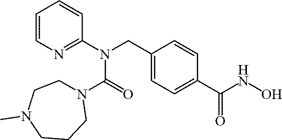 [SMILES: CN1CCCN(C(=O)N(Cc2ccc(C(=O)NO)cc2)c2ccccn2)CC1] | 385 | 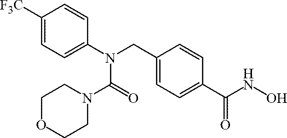 [SMILES: O=C(NO)c1ccc(CN(C(=O)N2CCOCC2)c2ccc(C(F)(F)F)cc2)cc1] |
| 377 | 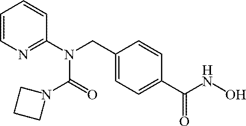 [SMILES: O=C(NO)c1ccc(CN(C(=O)N2CCC2)c2ccccn2)cc1] | 386 | 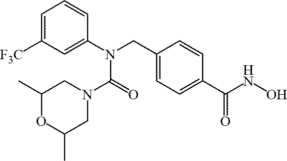 [SMILES: CC1CN(C(=O)N(Cc2ccc(C(=O)NO)cc2)c2cccc(C(F)(F)F)c2)CC(C)O1] |
| 379 | 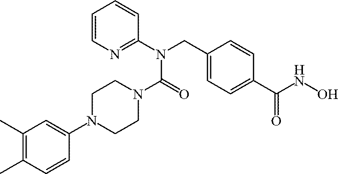 [SMILES: Cc1ccc(N2CCN(C(=O)N(Cc3ccc(C(=O)NO)cc3)c3ccccn3)CC2)cc1C] | 389 | 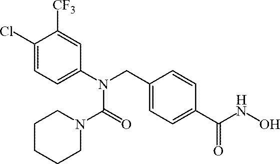 [SMILES: O=C(NO)c1ccc(CN(C(=O)N2CCCCC2)c2ccc(Cl)c(C(F)(F)F)c2)cc1] |
| 380 | 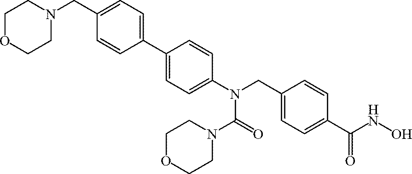 [SMILES: O=C(NO)c1ccc(CN(C(=O)N2CCOCC2)c2ccc(-c3ccc(CN4CCOCC4)cc3)cc2)cc1] | 390 | 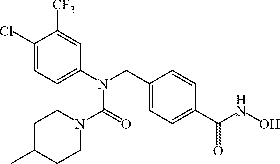 [SMILES: CC1CCN(C(=O)N(Cc2ccc(C(=O)NO)cc2)c2ccc(Cl)c(C(F)(F)F)c2)CC1] |
| 381 | 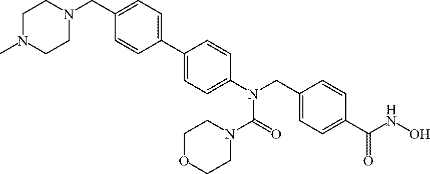 [SMILES: CN1CCN(Cc2ccc(-c3ccc(N(Cc4ccc(C(=O)NO)cc4)C(=O)N4CCOCC4)cc3)cc2)CC1] | 391 | 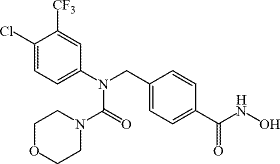 [SMILES: O=C(NO)c1ccc(CN(C(=O)N2CCOCC2)c2ccc(Cl)c(C(F)(F)F)c2)cc1] |
| 382 | 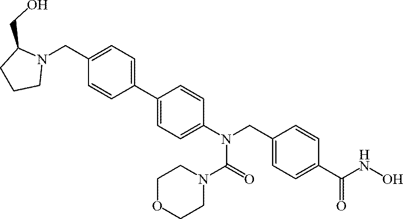 [SMILES: O=C(NO)c1ccc(CN(C(=O)N2CCOCC2)c2ccc(-c3ccc(CN4CCC[C@H]4CO)cc3)cc2)cc1] | 392 | 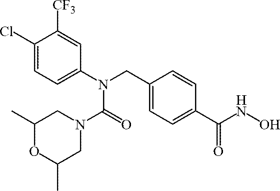 [SMILES: CC1CN(C(=O)N(Cc2ccc(C(=O)NO)cc2)c2ccc(Cl)c(C(F)(F)F)c2)CC(C)O1] |
| 383 | 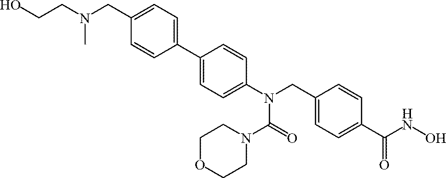 [SMILES: CN(CCO)Cc1ccc(-c2ccc(N(Cc3ccc(C(=O)NO)cc3)C(=O)N3CCOCC3)cc2)cc1] | 393 | 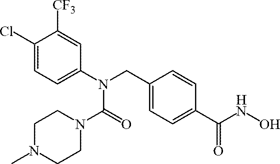 [SMILES: CN1CCN(C(=O)N(Cc2ccc(C(=O)NO)cc2)c2ccc(Cl)c(C(F)(F)F)c2)CC1] |
| 394 | 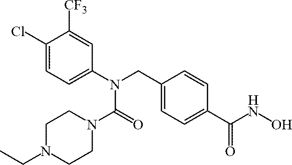 [SMILES: CCN1CCN(C(=O)N(Cc2ccc(C(=O)NO)cc2)c2ccc(Cl)c(C(F)(F)F)c2)CC1] | 401 | 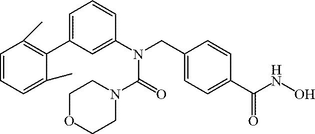 [SMILES: Cc1cccc(C)c1-c1cccc(N(Cc2ccc(C(=O)NO)cc2)C(=O)N2CCOCC2)c1] |
| 395 | 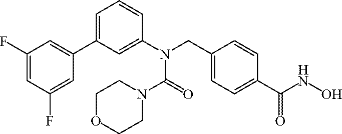 [SMILES: O=C(NO)c1ccc(CN(C(=O)N2CCOCC2)c2cccc(-c3cc(F)cc(F)c3)c2)cc1] | 402 | 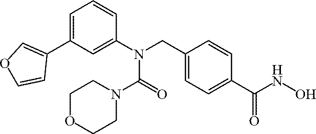 [SMILES: O=C(NO)c1ccc(CN(C(=O)N2CCOCC2)c2cccc(-c3ccoc3)c2)cc1] |
| 396 | 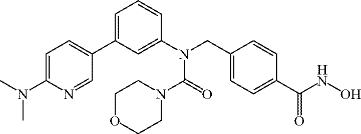 [SMILES: CN(C)c1ccc(-c2cccc(N(Cc3ccc(C(=O)NO)cc3)C(=O)N3CCOCC3)c2)cn1] | 403 | 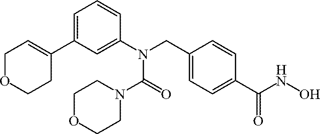 [SMILES: O=C(NO)c1ccc(CN(C(=O)N2CCOCC2)c2cccc(C3=CCOCC3)c2)cc1] |
| 397 | 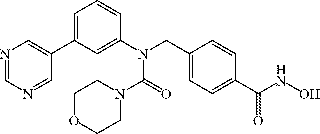 [SMILES: O=C(NO)c1ccc(CN(C(=O)N2CCOCC2)c2cccc(-c3cncnc3)c2)cc1] | 404 | 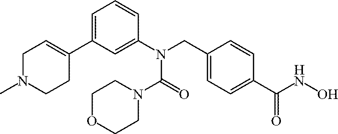 [SMILES: CN1CC=C(c2cccc(N(Cc3ccc(C(=O)NO)cc3)C(=O)N3CCOCC3)c2)CC1] |
| 398 | 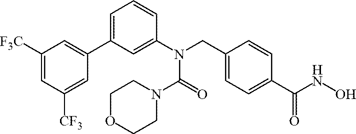 [SMILES: O=C(NO)c1ccc(CN(C(=O)N2CCOCC2)c2cccc(-c3cc(C(F)(F)F)cc(C(F)(F)F)c3)c2)cc1] | 405 | 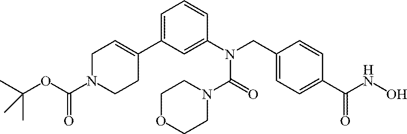 [SMILES: CC(C)(C)OC(=O)N1CC=C(c2cccc(N(Cc3ccc(C(=O)NO)cc3)C(=O)N3CCOCC3)c2)CC1] |
| 399 | 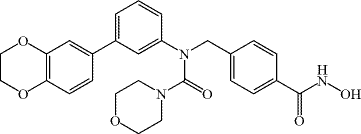 [SMILES: O=C(NO)c1ccc(CN(C(=O)N2CCOCC2)c2cccc(-c3ccc4c(c3)OCCO4)c2)cc1] | 413 | 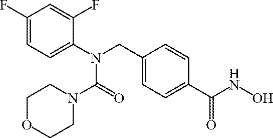 [SMILES: O=C(NO)c1ccc(CN(C(=O)N2CCOCC2)c2ccc(F)cc2F)cc1] |
| 400 | 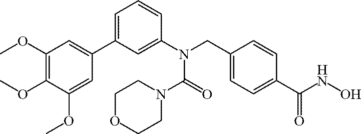 [SMILES: COc1cc(-c2cccc(N(Cc3ccc(C(=O)NO)cc3)C(=O)N3CCOCC3)c2)cc(OC)c1OC] | 414 | 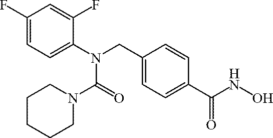 [SMILES: O=C(NO)c1ccc(CN(C(=O)N2CCCCC2)c2ccc(F)cc2F)cc1] |
| 415 | 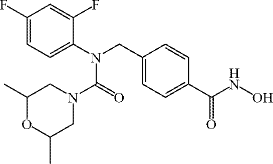 [SMILES: CC1CN(C(=O)N(Cc2ccc(C(=O)NO)cc2)c2ccc(F)cc2F)CC(C)O1] | 440 | 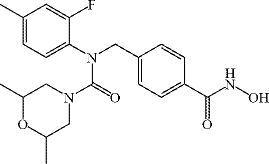 [SMILES: Cc1ccc(N(Cc2ccc(C(=O)NO)cc2)C(=O)N2CC(C)OC(C)C2)c(F)c1] |
| 416 | 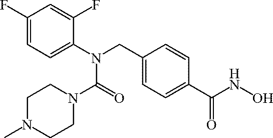 [SMILES: CN1CCN(C(=O)N(Cc2ccc(C(=O)NO)cc2)c2ccc(F)cc2F)CC1] | 441 | 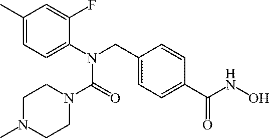 [SMILES: Cc1ccc(N(Cc2ccc(C(=O)NO)cc2)C(=O)N2CCN(C)CC2)c(F)c1] |
| 418 | 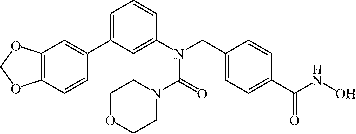 [SMILES: O=C(NO)c1ccc(CN(C(=O)N2CCOCC2)c2cccc(-c3ccc4c(c3)OCO4)c2)cc1] | 450 | 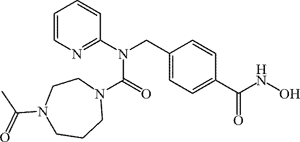 [SMILES: CC(=O)N1CCCN(C(=O)N(Cc2ccc(C(=O)NO)cc2)c2ccccn2)CC1] |
| 419 | 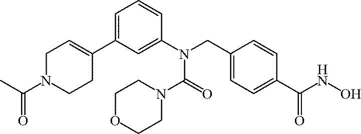 [SMILES: CC(=O)N1CC=C(c2cccc(N(Cc3ccc(C(=O)NO)cc3)C(=O)N3CCOCC3)c2)CC1] | 451 | 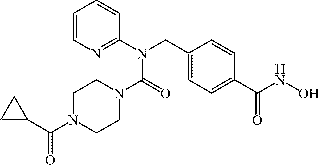 [SMILES: O=C(NO)c1ccc(CN(C(=O)N2CCN(C(=O)C3CC3)CC2)c2ccccn2)cc1] |
| 420 | 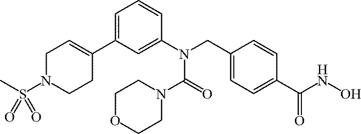 [SMILES: CS(=O)(=O)N1CC=C(c2cccc(N(Cc3ccc(C(=O)NO)cc3)C(=O)N3CCOCC3)c2)CC1] | 453 | 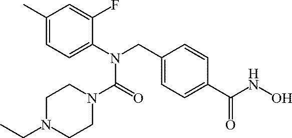 [SMILES: CCN1CCN(C(=O)N(Cc2ccc(C(=O)NO)cc2)c2ccc(C)cc2F)CC1] |
| 438 | 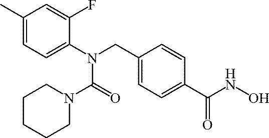 [SMILES: Cc1ccc(N(Cc2ccc(C(=O)NO)cc2)C(=O)N2CCCCC2)c(F)c1] | 454 | 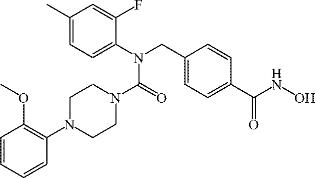 [SMILES: COc1ccccc1N1CCN(C(=O)N(Cc2ccc(C(=O)NO)cc2)c2ccc(C)cc2F)CC1] |
| 439 | 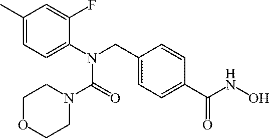 [SMILES: Cc1ccc(N(Cc2ccc(C(=O)NO)cc2)C(=O)N2CCOCC2)c(F)c1] | 455 | 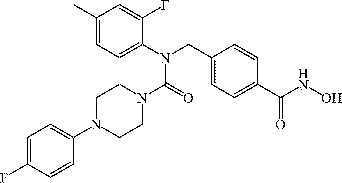 [SMILES: Cc1ccc(N(Cc2ccc(C(=O)NO)cc2)C(=O)N2CCN(c3ccc(F)cc3)CC2)c(F)c1] |
| 456 | 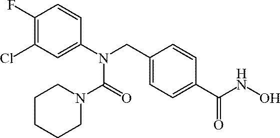 [SMILES: O=C(NO)c1ccc(CN(C(=O)N2CCCCC2)c2ccc(F)c(Cl)c2)cc1] | 463 | 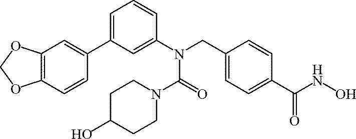 [SMILES: O=C(NO)c1ccc(CN(C(=O)N2CCC(O)CC2)c2cccc(-c3ccc4c(c3)OCO4)c2)cc1] |
| 457 | 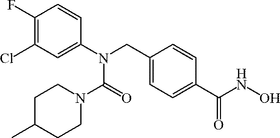 [SMILES: CC1CCN(C(=O)N(Cc2ccc(C(=O)NO)cc2)c2ccc(F)c(Cl)c2)CC1] | 464 | 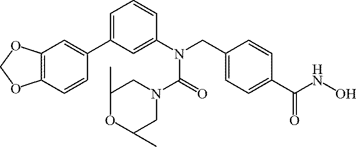 [SMILES: CC1CN(C(=O)N(Cc2ccc(C(=O)NO)cc2)c2cccc(-c3ccc4c(c3)OCO4)c2)CC(C)O1] |
| 458 | 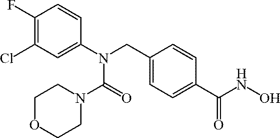 [SMILES: O=C(NO)c1ccc(CN(C(=O)N2CCOCC2)c2ccc(F)c(Cl)c2)cc1] | 465 | 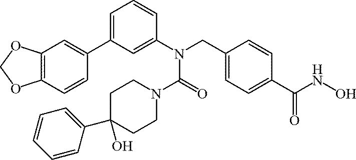 [SMILES: O=C(NO)c1ccc(CN(C(=O)N2CCC(O)(c3ccccc3)CC2)c2cccc(-c3ccc4c(c3)OCO4)c2)cc1] |
| 459 | 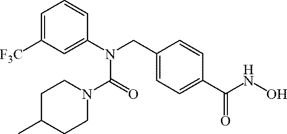 [SMILES: CC1CCN(C(=O)N(Cc2ccc(C(=O)NO)cc2)c2cccc(C(F)(F)F)c2)CC1] | 466 | 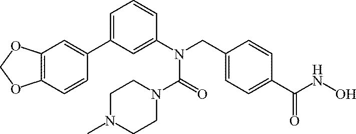 [SMILES: CN1CCN(C(=O)N(Cc2ccc(C(=O)NO)cc2)c2cccc(-c3ccc4c(c3)OCO4)c2)CC1] |
| 460 | 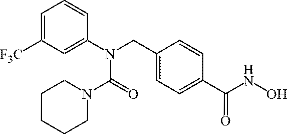 [SMILES: O=C(NO)c1ccc(CN(C(=O)N2CCCCC2)c2cccc(C(F)(F)F)c2)cc1] | 467 | 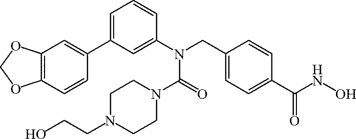 [SMILES: O=C(NO)c1ccc(CN(C(=O)N2CCN(CCO)CC2)c2cccc(-c3ccc4c(c3)OCO4)c2)cc1] |
| 461 | 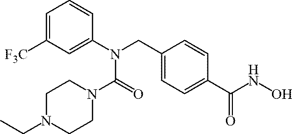 [SMILES: CCN1CCN(C(=O)N(Cc2ccc(C(=O)NO)cc2)c2cccc(C(F)(F)F)c2)CC1] | 468 | 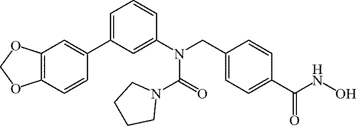 [SMILES: O=C(NO)c1ccc(CN(C(=O)N2CCCC2)c2cccc(-c3ccc4c(c3)OCO4)c2)cc1] |
| 462 | 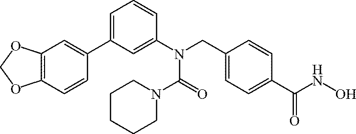 [SMILES: O=C(NO)c1ccc(CN(C(=O)N2CCCCC2)c2cccc(-c3ccc4c(c3)OCO4)c2)cc1] | 469 | 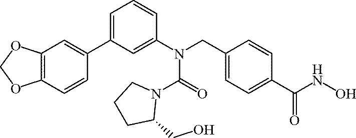 [SMILES: O=C(NO)c1ccc(CN(C(=O)N2CCC[C@H]2CO)c2cccc(-c3ccc4c(c3)OCO4)c2)cc1] |
| 470 | 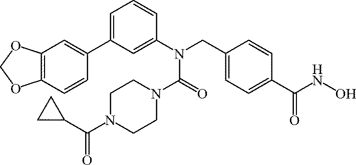 [SMILES: O=C(NO)c1ccc(CN(C(=O)N2CCN(C(=O)C3CC3)CC2)c2cccc(-c3ccc4c(c3)OCO4)c2)cc1] | 482 | 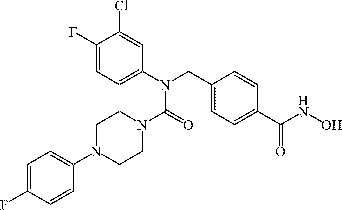 [SMILES: O=C(NO)c1ccc(CN(C(=O)N2CCN(c3ccc(F)cc3)CC2)c2ccc(F)c(Cl)c2)cc1] |
| 471 | 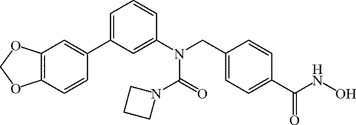 [SMILES: O=C(NO)c1ccc(CN(C(=O)N2CCC2)c2cccc(-c3ccc4c(c3)OCO4)c2)cc1] | 483 | 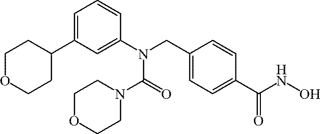 [SMILES: O=C(NO)c1ccc(CN(C(=O)N2CCOCC2)c2cccc(C3CCOCC3)c2)cc1] |
| 477 | 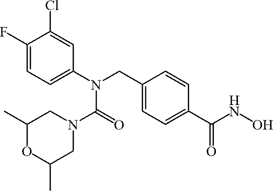 [SMILES: CC1CN(C(=O)N(Cc2ccc(C(=O)NO)cc2)c2ccc(F)c(Cl)c2)CC(C)O1] | 484 | 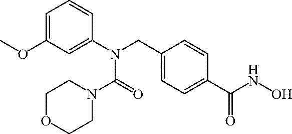 [SMILES: COc1cccc(N(Cc2ccc(C(=O)NO)cc2)C(=O)N2CCOCC2)c1] |
| 478 | 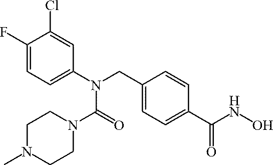 [SMILES: CN1CCN(C(=O)N(Cc2ccc(C(=O)NO)cc2)c2ccc(F)c(Cl)c2)CC1] | 485 | 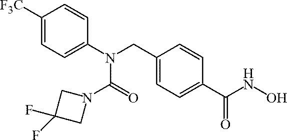 [SMILES: O=C(NO)c1ccc(CN(C(=O)N2CC(F)(F)C2)c2ccc(C(F)(F)F)cc2)cc1] |
| 479 | 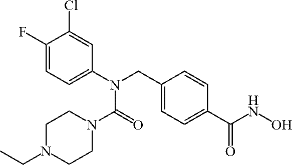 [SMILES: CCN1CCN(C(=O)N(Cc2ccc(C(=O)NO)cc2)c2ccc(F)c(Cl)c2)CC1] | 486 | 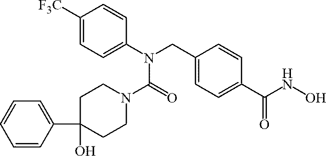 [SMILES: O=C(NO)c1ccc(CN(C(=O)N2CCC(O)(c3ccccc3)CC2)c2ccc(C(F)(F)F)cc2)cc1] |
| 480 | 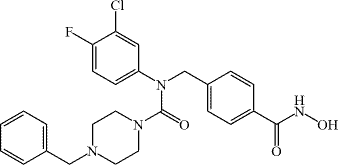 [SMILES: O=C(NO)c1ccc(CN(C(=O)N2CCN(Cc3ccccc3)CC2)c2ccc(F)c(Cl)c2)cc1] | 487 | 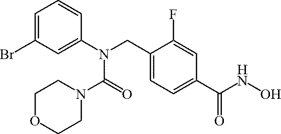 [SMILES: O=C(NO)c1ccc(CN(C(=O)N2CCOCC2)c2cccc(Br)c2)c(F)c1] |
| 481 | 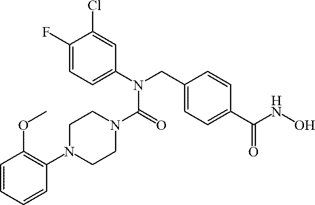 [SMILES: COc1ccccc1N1CCN(C(=O)N(Cc2ccc(C(=O)NO)cc2)c2ccc(F)c(Cl)c2)CC1] | 488 | 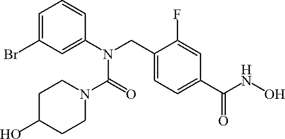 [SMILES: O=C(NO)c1ccc(CN(C(=O)N2CCC(O)CC2)c2cccc(Br)c2)c(F)c1] |
| 489 | 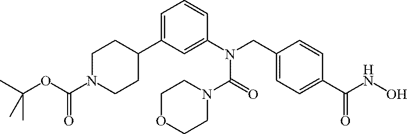 [SMILES: CC(C)(C)OC(=O)N1CCC(c2cccc(N(Cc3ccc(C(=O)NO)cc3)C(=O)N3CCOCC3)c2)CC1] | 496 | 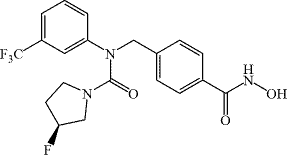 [SMILES: O=C(NO)c1ccc(CN(C(=O)N2CC[C@H](F)C2)c2cccc(C(F)(F)F)c2)cc1] |
| 490 | 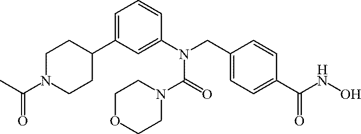 [SMILES: CC(=O)N1CCC(c2cccc(N(Cc3ccc(C(=O)NO)cc3)C(=O)N3CCOCC3)c2)CC1] | 497 | 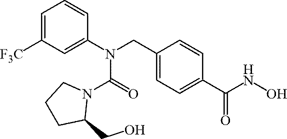 [SMILES: O=C(NO)c1ccc(CN(C(=O)N2CCC[C@@H]2CO)c2cccc(C(F)(F)F)c2)cc1] |
| 491 | 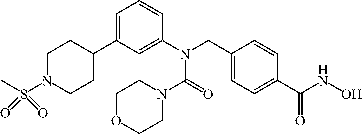 [SMILES: CS(=O)(=O)N1CCC(c2cccc(N(Cc3ccc(C(=O)NO)cc3)C(=O)N3CCOCC3)c2)CC1] | 498 | 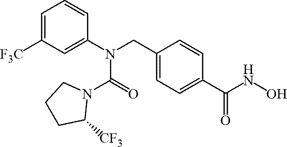 [SMILES: O=C(NO)c1ccc(CN(C(=O)N2CCC[C@H]2C(F)(F)F)c2cccc(C(F)(F)F)c2)cc1] |
| 492 | 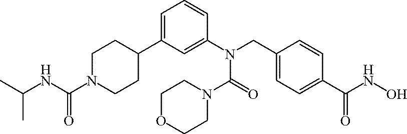 [SMILES: CC(C)NC(=O)N1CCC(c2cccc(N(Cc3ccc(C(=O)NO)cc3)C(=O)N3CCOCC3)c2)CC1] | 499 | 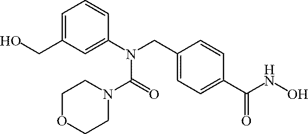 [SMILES: O=C(NO)c1ccc(CN(C(=O)N2CCOCC2)c2cccc(CO)c2)cc1] |
| 493 | 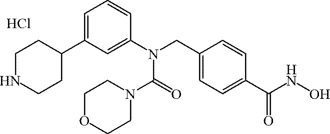 [SMILES: Cl ; O=C(NO)c1ccc(CN(C(=O)N2CCOCC2)c2cccc(C3CCNCC3)c2)cc1] | 500 | 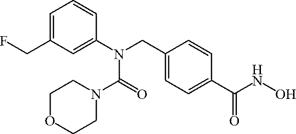 [SMILES: O=C(NO)c1ccc(CN(C(=O)N2CCOCC2)c2cccc(CF)c2)cc1] |
| 494 | 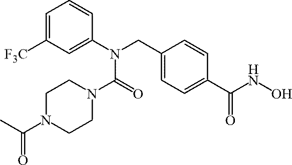 [SMILES: CC(=O)N1CCN(C(=O)N(Cc2ccc(C(=O)NO)cc2)c2cccc(C(F)(F)F)c2)CC1] | 511 | 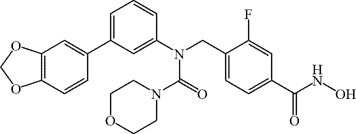 [SMILES: O=C(NO)c1ccc(CN(C(=O)N2CCOCC2)c2cccc(-c3ccc4c(c3)OCO4)c2)c(F)c1] |
| 495 | 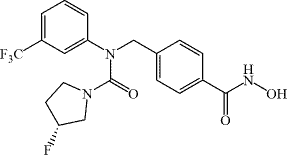 [SMILES: O=C(NO)c1ccc(CN(C(=O)N2CC[C@@H](F)C2)c2cccc(C(F)(F)F)c2)cc1] | 512 | 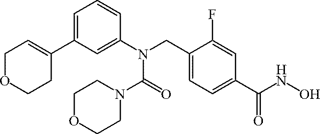 [SMILES: O=C(NO)c1ccc(CN(C(=O)N2CCOCC2)c2cccc(C3=CCOCC3)c2)c(F)c1] |
| 513 | 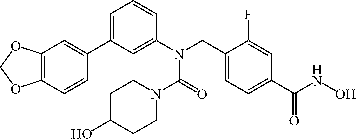 [SMILES: O=C(NO)c1ccc(CN(C(=O)N2CCC(O)CC2)c2cccc(-c3ccc4c(c3)OCO4)c2)c(F)c1] | 529 | 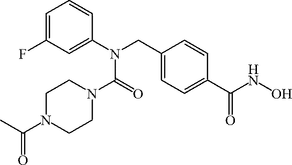 [SMILES: CC(=O)N1CCN(C(=O)N(Cc2ccc(C(=O)NO)cc2)c2cccc(F)c2)CC1] |
| 514 | 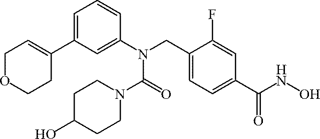 [SMILES: O=C(NO)c1ccc(CN(C(=O)N2CCC(O)CC2)c2cccc(C3=CCOCC3)c2)c(F)c1] | 530 | 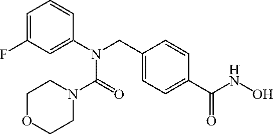 [SMILES: O=C(NO)c1ccc(CN(C(=O)N2CCOCC2)c2cccc(F)c2)cc1] |
| 517 | 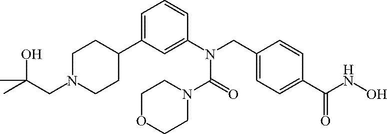 [SMILES: CC(C)(O)CN1CCC(c2cccc(N(Cc3ccc(C(=O)NO)cc3)C(=O)N3CCOCC3)c2)CC1] | 531 | 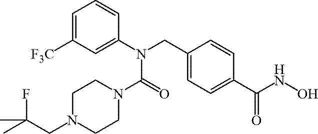 [SMILES: CC(C)(F)CN1CCN(C(=O)N(Cc2ccc(C(=O)NO)cc2)c2cccc(C(F)(F)F)c2)CC1] |
| 518 | 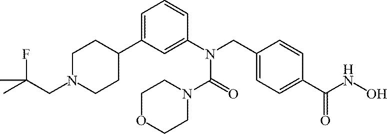 [SMILES: CC(C)(F)CN1CCC(c2cccc(N(Cc3ccc(C(=O)NO)cc3)C(=O)N3CCOCC3)c2)CC1] | 532 | 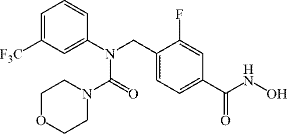 [SMILES: O=C(NO)c1ccc(CN(C(=O)N2CCOCC2)c2cccc(C(F)(F)F)c2)c(F)c1] |
| 520 | 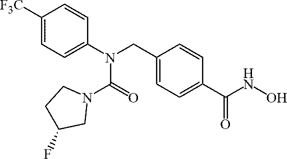 [SMILES: O=C(NO)c1ccc(CN(C(=O)N2CC[C@@H](F)C2)c2ccc(C(F)(F)F)cc2)cc1] | 533 | 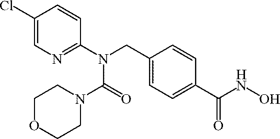 [SMILES: O=C(NO)c1ccc(CN(C(=O)N2CCOCC2)c2ccc(Cl)cn2)cc1] |
| 521 | 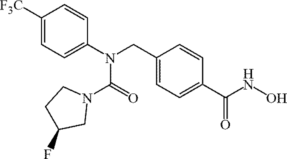 [SMILES: O=C(NO)c1ccc(CN(C(=O)N2CC[C@H](F)C2)c2ccc(C(F)(F)F)cc2)cc1] | 543 | 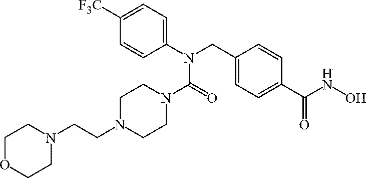 [SMILES: O=C(NO)c1ccc(CN(C(=O)N2CCN(CCN3CCOCC3)CC2)c2ccc(C(F)(F)F)cc2)cc1] |
| 522 | 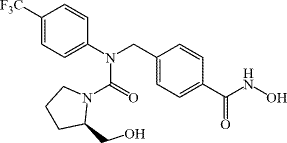 [SMILES: O=C(NO)c1ccc(CN(C(=O)N2CCC[C@@H]2CO)c2ccc(C(F)(F)F)cc2)cc1] | 544 | 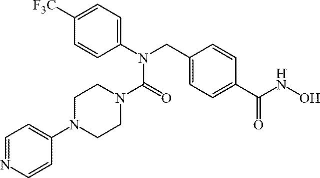 [SMILES: O=C(NO)c1ccc(CN(C(=O)N2CCN(c3ccncc3)CC2)c2ccc(C(F)(F)F)cc2)cc1] |
| 545 | 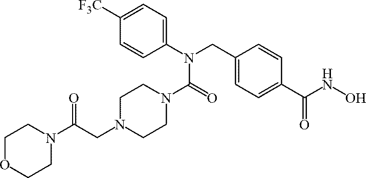 [SMILES: O=C(NO)c1ccc(CN(C(=O)N2CCN(CC(=O)N3CCOCC3)CC2)c2ccc(C(F)(F)F)cc2)cc1] | 716 | 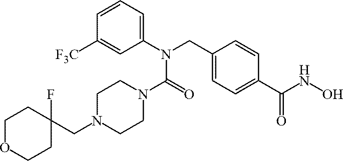 [SMILES: O=C(NO)c1ccc(CN(C(=O)N2CCN(CC3(F)CCOCC3)CC2)c2cccc(C(F)(F)F)c2)cc1] |
| 577 | 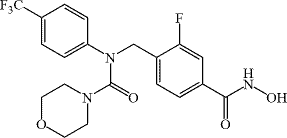 [SMILES: O=C(NO)c1ccc(CN(C(=O)N2CCOCC2)c2ccc(C(F)(F)F)cc2)c(F)c1] | 717 | 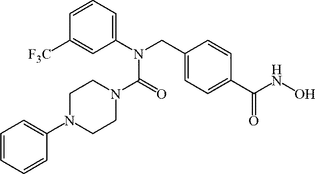 [SMILES: O=C(NO)c1ccc(CN(C(=O)N2CCN(c3ccccc3)CC2)c2cccc(C(F)(F)F)c2)cc1] |
| 578 | 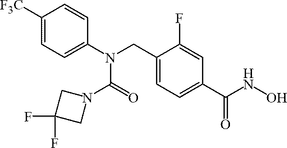 [SMILES: O=C(NO)c1ccc(CN(C(=O)N2CC(F)(F)C2)c2ccc(C(F)(F)F)cc2)c(F)c1] | 718 | 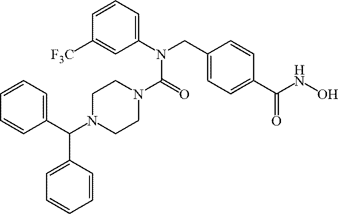 [SMILES: O=C(NO)c1ccc(CN(C(=O)N2CCN(C(c3ccccc3)c3ccccc3)CC2)c2cccc(C(F)(F)F)c2)cc1] |
| 580 | 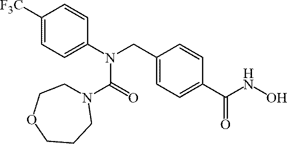 [SMILES: O=C(NO)c1ccc(CN(C(=O)N2CCCOCC2)c2ccc(C(F)(F)F)cc2)cc1] | 765 | 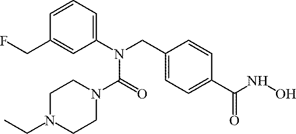 [SMILES: CCN1CCN(C(=O)N(Cc2ccc(C(=O)NO)cc2)c2cccc(CF)c2)CC1] |
| 651 | 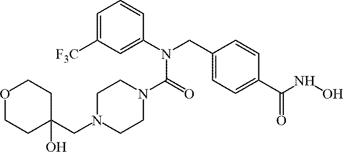 [SMILES: O=C(NO)c1ccc(CN(C(=O)N2CCN(CC3(O)CCOCC3)CC2)c2cccc(C(F)(F)F)c2)cc1] | 766 | 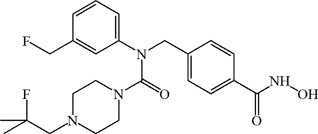 [SMILES: CC(C)(F)CN1CCN(C(=O)N(Cc2ccc(C(=O)NO)cc2)c2cccc(CF)c2)CC1] |
| 652 | 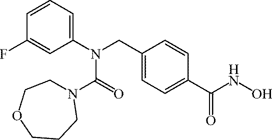 [SMILES: O=C(NO)c1ccc(CN(C(=O)N2CCCOCC2)c2cccc(F)c2)cc1] | 771 | 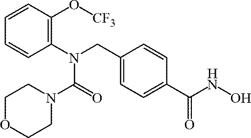 [SMILES: O=C(NO)c1ccc(CN(C(=O)N2CCOCC2)c2ccccc2OC(F)(F)F)cc1] |
| 683 | 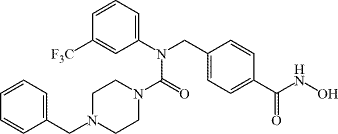 [SMILES: O=C(NO)c1ccc(CN(C(=O)N2CCN(Cc3ccccc3)CC2)c2cccc(C(F)(F)F)c2)cc1] | 772 | 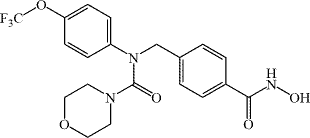 [SMILES: O=C(NO)c1ccc(CN(C(=O)N2CCOCC2)c2ccc(OC(F)(F)F)cc2)cc1] |
| 684 | 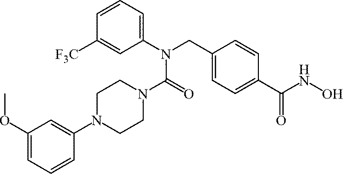 [SMILES: COc1cccc(N2CCN(C(=O)N(Cc3ccc(C(=O)NO)cc3)c3cccc(C(F)(F)F)c3)CC2)c1] | | |
| 773 | 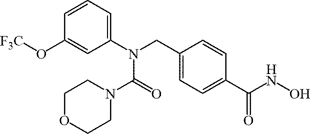 [SMILES: O=C(NO)c1ccc(CN(C(=O)N2CCOCC2)c2cccc(OC(F)(F)F)c2)cc1] | 801 | 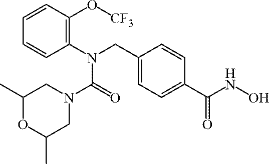 [SMILES: CC1CN(C(=O)N(Cc2ccc(C(=O)NO)cc2)c2ccccc2OC(F)(F)F)CC(C)O1] |
| 774 | 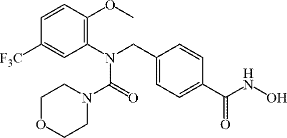 [SMILES: COc1ccc(C(F)(F)F)cc1N(Cc1ccc(C(=O)NO)cc1)C(=O)N1CCOCC1] | 802 | 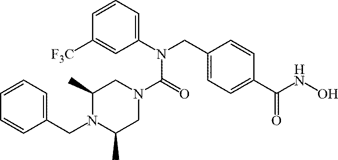 [SMILES: C[C@@H]1CN(C(=O)N(Cc2ccc(C(=O)NO)cc2)c2cccc(C(F)(F)F)c2)C[C@H](C)N1Cc1ccccc1] |
| 776 | 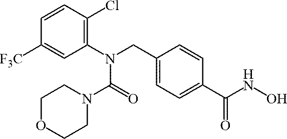 [SMILES: O=C(NO)c1ccc(CN(C(=O)N2CCOCC2)c2cc(C(F)(F)F)ccc2Cl)cc1] | 803 | 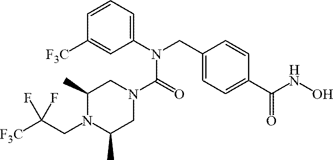 [SMILES: C[C@@H]1CN(C(=O)N(Cc2ccc(C(=O)NO)cc2)c2cccc(C(F)(F)F)c2)C[C@H](C)N1CC(F)(F)C(F)(F)F] |
| 778 | 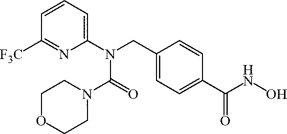 [SMILES: O=C(NO)c1ccc(CN(C(=O)N2CCOCC2)c2cccc(C(F)(F)F)n2)cc1] | 826 | 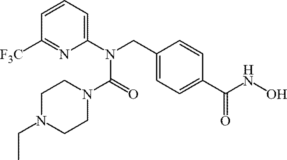 [SMILES: CCN1CCN(C(=O)N(Cc2ccc(C(=O)NO)cc2)c2cccc(C(F)(F)F)n2)CC1] |
| 791 | 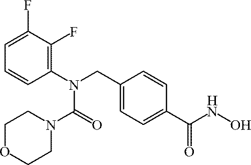 [SMILES: O=C(NO)c1ccc(CN(C(=O)N2CCOCC2)c2cccc(F)c2F)cc1] | 827 | 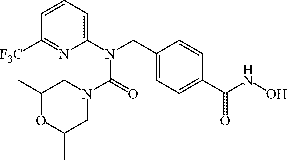 [SMILES: CC1CN(C(=O)N(Cc2ccc(C(=O)NO)cc2)c2cccc(C(F)(F)F)n2)CC(C)O1] |
| 797 | 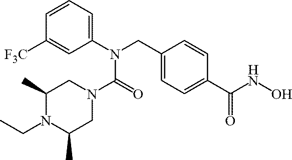 [SMILES: CCN1[C@H](C)CN(C(=O)N(Cc2ccc(C(=O)NO)cc2)c2cccc(C(F)(F)F)c2)C[C@@H]1C] | 828 | 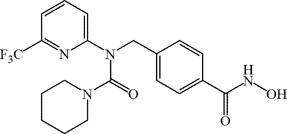 [SMILES: O=C(NO)c1ccc(CN(C(=O)N2CCCCC2)c2cccc(C(F)(F)F)n2)cc1] |
| 800 | 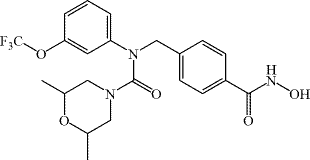 [SMILES: CC1CN(C(=O)N(Cc2ccc(C(=O)NO)cc2)c2cccc(OC(F)(F)F)c2)CC(C)O1] | 829 | 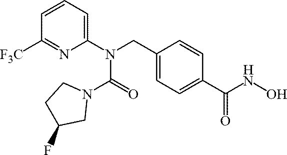 [SMILES: O=C(NO)c1ccc(CN(C(=O)N2CC[C@H](F)C2)c2cccc(C(F)(F)F)n2)cc1] |

According to a specific embodiment of the present invention, the compound represented by the above chemical formula I may be a compound described in a following table.

| Compound | Structure | Compound | Structure |
|---|---|---|---|
| 374 | 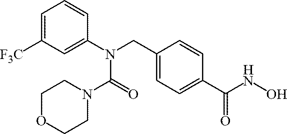 [SMILES: O=C(NO)c1ccc(CN(C(=O)N2CCOCC2)c2cccc(C(F)(F)F)c2)cc1] | 413 | 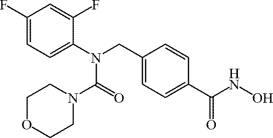 [SMILES: O=C(NO)c1ccc(CN(C(=O)N2CCOCC2)c2ccc(F)cc2F)cc1] |
| 458 | 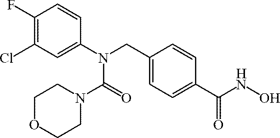 [SMILES: O=C(NO)c1ccc(CN(C(=O)N2CCOCC2)c2ccc(F)c(Cl)c2)cc1] | 484 | 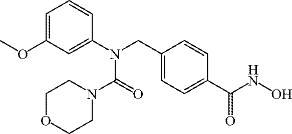 [SMILES: COc1cccc(N(Cc2ccc(C(=O)NO)cc2)C(=O)N2CCOCC2)c1] |
| 530 | 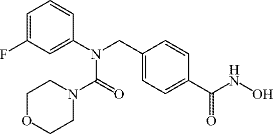 [SMILES: O=C(NO)c1ccc(CN(C(=O)N2CCOCC2)c2cccc(F)c2)cc1] | 652 | 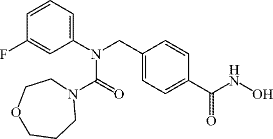 [SMILES: O=C(NO)c1ccc(CN(C(=O)N2CCCOCC2)c2cccc(F)c2)cc1] |

In the present invention, the compound represented by the above chemical formula I may be prepared by a method disclosed in Korean Unexamined Patent Publication No. 2014-0128886, but is not limited thereto.

In the present invention, the term “pharmaceutically acceptable” may refer to the one which is physiologically acceptable and does not conventionally cause an allergic response such as gastrointestinal disturbance, dizziness, etc., or other responses similar thereto, when being administered into individuals.

The pharmaceutically acceptable salt of the present invention may be prepared by a conventional method known to those skilled in the art.

The pharmaceutically acceptable salts of the present invention may include, for example, inorganic ion salts prepared from calcium, potassium, sodium, magnesium, etc.; inorganic acid salts prepared from hydrochloric acid, nitric acid, phosphoric acid, bromic acid, iodic acid, perchloric acid, sulfuric acid hydroiodic acid, etc.; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbric acid, carbonic acid, vanillic acid, etc.; sulfonic acid salts prepared from methanesulfonic acid, ethanesulfonic acid, benzenesulfonic acid, p-toluenesulfonic acid, naphthalenesulfonic acid and the like; amino acid salts prepared from glycine, arginine, lysine, etc.; amine salts prepared from trimethylamine, triethylamine, ammonia, pyridine, picoline, etc.; and the like, but are not limited thereto. In the present invention, preferable salts may include hydrochloric acid, trifluoroacetic acid, citric acid, bromic acid, maleic acid, phosphoric acid, sulfuric acid and tartaric acid.

In the present invention, the term “chronic obstructive pulmonary disease (COPD)” may refer to a disease which is accompanied by irreversible occlusion in the airway and results from pathological changes in the bronchioles and the lung parenchyma caused by airway and lung parenchymal inflammation, and include chronic obstructive bronchitis, chronic bronchiolitis, pulmonary emphysema (pulmonary parenchymal destruction) and the like.

In the present invention, the term “prevention” may refer to all the acts, which inhibit or delay the occurrence of a disease by administering the compound of the chemical formula I of the present invention, optical isomers thereof or pharmaceutically acceptable salts thereof.

In the present invention, the term “treatment” may refer to all the acts, by which a symptom of an individual likely to develop or suffering from a disease gets better or takes a favorable turn by administering the compound of the chemical formula I of the present invention, optical isomers thereof or pharmaceutically acceptable salts thereof.

The compound represented by the chemical formula I of the present invention, optical isomers thereof or pharmaceutically acceptable salts thereof may be useful for preventing or treating chronic obstructive pulmonary disease.

A pharmaceutical composition comprising the compound represented by the chemical formula I of the present invention, optical isomers thereof or pharmaceutically acceptable salts thereof as an effective ingredient may be useful for preventing or treating chronic obstructive pulmonary disease.

In this regard, according to one specific embodiment of the present invention, it was confirmed that the compound represented by the chemical formula I of the present invention, optical isomers thereof or pharmaceutically acceptable salts thereof inhibits infiltration of immune cells (FIG. 2), inhibits infiltration of inflammatory cells by decreasing the number of total cells, macrophages and neutrophils (FIG. 3), and decreases expression of inflammatory cytokines, specifically IL-6, IFN-y, MCP-1, and TNF-α (FIG. 4).

In addition, it was confirmed that the above compound remarkably increases expression of E-Cad, an epithelial marker protein, remarkably decreases expression of N-Cad, a mesenchymal marker protein to inhibit TGF-β1-induced EMT (FIG. 5), and decreases expression of FN-EDA, a major ECM protein (FIG. 6).

The pharmaceutical composition of the present invention may show an effect of preventing or treating chronic obstructive pulmonary disease at a level that may be considered to be similar to, substantially the same as, or better than a conventionally known composition for treating COPD.

The pharmaceutical composition of the present invention may further comprise at least one type of a pharmaceutically acceptable carrier, in addition to the compound represented by the above chemical formula I, optical isomers thereof or pharmaceutically acceptable salts thereof. The pharmaceutically acceptable carrier may be the one that is conventionally used in the art, specifically including, but not limited thereto, lactose, dextrose, sucrose, sorbitol, mannitol, starch, acacia rubber, calcium phosphate, alginate, gelatin, calcium silicate, microcrystalline cellulose, polyvinyl pyrrolidine, cellulose, water, syrup, methyl cellulose, methyl hydroxybenzoate, propyl hydroxybenzoate, talc, magnesium stearate or mineral oil. The pharmaceutical composition of the present invention may further comprise lubricant, humectant, a sweetening agent, a flavoring agent, emulsifier, a suspending agent, preservative, a dispersing agent, a stabilizing agent, etc., in addition to the above ingredients. In addition, the pharmaceutical composition of the present invention may be formulated into an oral dosage form such as tablet, powder, granule, pill, capsule, suspension, emulsion, liquid for internal use, oiling agent, syrup, etc., as well as a form of external preparation, suppository and sterile solution for injection by using a pharmaceutically acceptable carrier and excipient, and thus may be prepared in a unit dose form or prepared by being inserted into a multi-dose container. Such preparations may be prepared according to a conventional method used for formulation in the art or a method disclosed in Remington’s Pharmaceutical Science (19^{th} ed., 1995), and may be formulated into various preparations depending on each disease or ingredient.

As a non-limiting example of preparations for oral administration using the pharmaceutical composition of the present invention, there may be tablets, troches, lozenges, water-soluble suspensions, oil suspensions, prepared powders, granules, emulsions, hard capsules, soft capsules, syrups, elixirs or the like. To formulate the pharmaceutical composition of the present invention into preparations for oral administration, the followings may be used: binders such as lactose, saccharose, sorbitol, mannitol, starch, amylopectin, cellulose, gelatin or the like; excipients such as dicalcium phosphate, etc.; disintegrants such as maize starch, sweet potato starch or the like; lubricants such as magnesium stearate, calcium stearate, sodium stearyl fumarate, polyethylene glycol wax or the like; etc., in which sweetening agents, flavoring agents, syrups, etc. may also be used. Furthermore, in case of the capsules, liquid carriers such as fatty oil, etc. may be further used in addition to the above-mentioned materials.

As a non-limiting example of parenteral preparations using the pharmaceutical composition of the present invention, there may be injectable solutions, suppositories, powders for respiratory inhalation, aerosols for spray, ointments, powders for application, oils, creams, etc. To formulate the pharmaceutical composition of the present invention into preparations for parenteral administration, the followings may be used: sterilized aqueous solutions, nonaqueous solvents, suspensions, emulsions, freeze-dried preparations, external preparations, etc. As said nonaqueous solvents and suspensions, the followings may be used, but not limited thereto: propylene glycol, polyethylene glycol, vegetable oils such as olive oil, injectable esters such as ethyl oleate, etc.

The pharmaceutical composition of the present invention may be subjected to oral administration or parenteral administration depending on an intended method, preferably oral administration, but is not limited thereto.

A daily dosage of the compound represented by the chemical formula I of the present invention, optical isomers thereof or pharmaceutically acceptable salts thereof may be specifically about 0.1 to 10,000 mg/kg, about 1 to 8,000 mg/kg, about 5 to 6,000 mg/kg, or about 10 to 4,000 mg/kg, and more specifically about 50 to 2,000 mg/kg, but is not limited thereto, and may be also administered once a day or several times a day by dividing the daily dosage of the compound.

A pharmaceutically effective dose and an effective dosage of the pharmaceutical composition of the present invention may vary depending on a method for formulating the pharmaceutical composition, an administration mode, an administration time and/or an administration route, etc., and may be diversified according to various factors including a type and degree of reaction to be achieved by administration of the pharmaceutical composition, a type of an individual for administration, the individual’s age, weight, general health condition, disease symptom or severity, gender, diet and excretion, ingredients of other drug compositions to be used for the corresponding individual at the same time or different times, etc., as well as other similar factors well known in a pharmaceutical field, and those skilled in the art may easily determine and prescribe an effective dosage for intended treatment.

The pharmaceutical composition of the present invention may be administered once a day or divided into several times a day by dividing the daily dosage of the composition. The pharmaceutical composition of the present invention may be administered as an individual therapeutic agent or in combination with other therapeutic agents, and may be administered sequentially or simultaneously with a conventional therapeutic agent. Considering all the above factors, the pharmaceutical composition of the present invention may be administered in such an amount that a maximum effect may be achieved by a minimum amount without a side effect, and such amount may be easily determined by those skilled in the art to which the present invention pertains.

The pharmaceutical composition of the present invention may show an excellent effect even when solely used, but may be further used in combination with various methods such as hormone therapy, drug treatment, etc. in order to increase a therapeutic efficiency.

The present invention provides a method for preventing or treating chronic obstructive pulmonary disease, including administering said pharmaceutical composition into an individual.

The present invention provides a method for preventing or treating chronic obstructive pulmonary disease, including administering a therapeutically effective amount of a compound represented by the chemical formula I, optical isomers thereof or pharmaceutically acceptable salts thereof into an individual.

Said terms “chronic obstructive pulmonary disease,” “prevention” and “treatment” may be the same as described above.

In the present invention, the term “administration” may refer to introducing a predetermined substance into an individual by an appropriate method.

In the present invention, the term “individual” may refer to all the animals such as rats, mice, livestock, etc., including humans, who are likely to develop or have already developed chronic obstructive pulmonary disease, and specifically refer to mammals including humans, but is not limited thereto.

A method for preventing or treating chronic obstructive pulmonary disease (COPD) according to the present invention may refer to administering a therapeutically effective amount of said pharmaceutical composition.

In the present invention, the term “therapeutically effective amount” may refer to an amount enough to treat a disease at a reasonable risk/benefit ratio applicable to medical treatment and not to cause a side effect, and may be determined by those skilled in the art according to factors including a patient’s gender, age, weight and health condition, a type of disease, severity, activity of a drug, sensitivity to a drug, an administration method, an administration time, an administration route, excretion rate, a treatment period, a drug combined or concurrently used, as well as other factors well known in a pharmaceutical field. It is preferable that a specific therapeutically effective amount is to be differently applied to each certain patient depending on various factors including a type and degree of reaction to be achieved therefrom, a specific composition including a presence of other preparations used in some cases, a patient’s age, weight, general health condition, gender and diet, an administration time, an administration route, a secretion rate of the composition, a treatment period and a drug used together with the specific composition or simultaneously therewith, as well as other similar factors well known in a pharmaceutical field.

The method for preventing or treating chronic obstructive pulmonary disease according to the present invention may include not only dealing with the disease per se before expression of its symptoms, but also inhibiting or avoiding such symptoms by administering the compound represented by the above chemical formula I, optical isomers thereof or pharmaceutically acceptable salts thereof. In managing diseases, a preventive or therapeutic dose of a certain active ingredient may vary depending on characteristics and severity of the diseases or conditions, and a route in which the active ingredient is administered. A dose and a frequency thereof may vary depending on an individual patient’s age, weight and reactions. A suitable dose and usage may be easily selected by those skilled in the art, naturally considering such factors. In addition, the method for preventing or treating chronic obstructive pulmonary disease according to the present invention may further comprise administering a therapeutically effective amount of an additional active agent, which is helpful in preventing or treating the disease, along with the compound represented by the above chemical formula I, optical isomers thereof or pharmaceutically acceptable salts thereof, and the additional active agent may show a synergy effect or an additive effect together with the compound represented by the above chemical formula I, optical isomers thereof or pharmaceutically acceptable salts thereof.

The present invention provides a use of a compound represented by the chemical formula I, optical isomers thereof or pharmaceutically acceptable salts thereof for preventing or treating chronic obstructive pulmonary disease.

The present invention provides a use of a compound represented by the chemical formula I, optical isomers thereof or pharmaceutically acceptable salts thereof in preparation of a medicament for preventing or treating chronic obstructive pulmonary disease.

The present invention provides a use of the pharmaceutical composition of the present invention in preparation of a medicament for preventing or treating chronic obstructive pulmonary disease.

Said terms “chronic obstructive pulmonary disease,” “prevention” and “treatment” may be the same as described above.

For preparation of the medicament, the compound represented by the chemical formula I of the present invention, optical isomers thereof or pharmaceutically acceptable salts thereof may be mixed with pharmaceutically acceptable adjuvants, diluents, carriers, etc., and may be prepared into a complex preparation together with other active agents, thus providing a synergy action.

Matters mentioned in the pharmaceutical composition, treatment method and use of the present invention are applied the same, if not contradictory to each other.

### Advantageous Effects

A compound represented by a chemical formula I of the present invention, optical isomers thereof or pharmaceutically acceptable salts thereof shows an excellent inhibitory effect on the infiltration of immune cells and the infiltration of inflammatory cells, and an antiinflammatory effect in a mouse with induced COPD, as well as an inhibitory effect on TGFβ1-induced EMT and the expression of FN-EDA, which is a major ECM protein, and thus can be useful in preventing or treating chronic obstructive pulmonary disease.

### BRIEF DESCRIPTION OF THE DRAWINGS

FIG. 1 is a view showing reference images for determining a degree of cellular infiltration in lung tissues.

FIG. 2 is a graph showing scores measured by observing a degree of cellular infiltration in groups dosed with a comparative composition and compositions of embodiments, respectively.

FIG. 3 is a graph showing the number of total cells, macrophages, and neutrophils in groups dosed with a comparative composition and compositions of embodiments, respectively.

FIG. 4 is a graph showing an expression level of inflammatory cytokines (IL-6, IFN-γ, MCP-1, and TNF-α) in groups dosed with a comparative composition and compositions of embodiments, respectively.

FIG. 5 is a graph showing an expression level of an epithelial marker protein (E-Cad) and a mesenchymal marker protein (N-Cad) in groups treated with a comparative composition and example compositions, respectively.

FIG. 6 is a graph showing an expression level of a main ECM protein (FN-EDA) in groups treated with a comparative composition and compositions of embodiments, respectively.

### BEST MODE FOR INVENTION

Hereinafter, the present invention will be described in more detail through exemplary embodiments. These exemplary embodiments are provided only for the purpose of illustrating the present invention, and thus it will be apparent to those skilled in the art that the scope of the present invention is not limited thereto.

### Preparation Example 1. Synthesis of N-(4-(Hydroxycarbamoyl)Benzyl)-N-(3-(Trifluoromethyl)Phenyl)Morpholine-4-Carboxamide [Compound 374]

[Step 1] Synthesis of methyl 4-((3-(trifluoromethyl)phenylamino)methyl)benzoate)

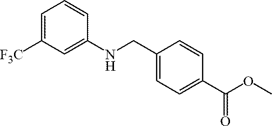 [SMILES: COC(=O)c1ccc(CNc2cccc(C(F)(F)F)c2)cc1]

A 3-(trifluoromethyl)benzenamine (0.30 g, 1.84 mmol) and potassium carbonate (0.76 g, 5.53 mmol) were dissolved in dimethylformamide (DMF, 5 mL), after which methyl 4-(bromomethyl)benzoate (0.42 g, 1.84 mmol) was inserted. A resulting solution was subjected to reaction at room temperature for a day and diluted with ethyl acetate. A reactant was washed with water and saturated sodium chloride aqueous solution, then dried and filtered with anhydrous magnesium sulfate, and then concentrated under reduced pressure. A residue was purified via column chromatography (silicon dioxide; ethyl acetate/hexane = 20%), such that a title compound (0.37 g, 65%) was obtained.

¹H NMR (400 MHz, DMSO-d₆) δ 7.93 (d, 2 H, J = 8.3 Hz), 7.49 (d, 2 H, J = 8.3 Hz), 7.24 (t, ¹ H, J = 7.9 Hz), 6.88-6.78 (m, 4 H), 4.42 (d, 2 H, J = 6.1 Hz), 3.83 (s, 3H), MS (ESI) m/z 310 (M⁺ + H).

[Step 2] Synthesis of methyl 4-((((4-nitrophenoxy)carbonyl)(3-(trifluoromethyl)phenyl)amino)methyl)benzoate

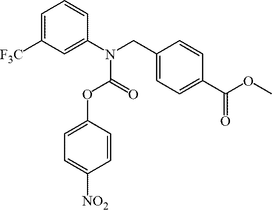 [SMILES: COC(=O)c1ccc(CN(C(=O)Oc2ccc([N+](=O)[O-])cc2)c2cccc(C(F)(F)F)c2)cc1]

Methyl 4-((3-(trifluoromethyl)phenylamino)methyl)benzoate (0.26 g, 0.82 mmol) and 4-nitrophenyl carbonochloridate (0.33 g, 1.65 mmol) were dissolved in acetonitrile (10 mL), after which potassium carbonate (0.34 g, 2.47 mmol) was inserted. A resulting solution was subjected to reaction at room temperature for a day and diluted with ethyl acetate. A reactant was washed with saturated sodium chloride aqueous solution, then dried and filtered with anhydrous sodium sulfate, and then concentrated under reduced pressure. A residue was purified via column chromatography (silicon dioxide; ethyl acetate/hexane = 20%), such that a title compound (0.35 g, 89%) was obtained in a colorless oil form.

¹H NMR (400 MHz, CDCl₃) δ 8.20 (d, 2 H, J = 10.2 Hz), 8.01 (d, 2 H, J = 7.8 Hz), 7.56-7.46 (m, 3H), 7.35 (d, 3 H, J = 8.0 Hz), 7.26 (d, 2 H, J = 8.1 Hz), 5.01 (bs, 2H), 3.90 (s, 3H).

[Step 3] Synthesis of methyl 4-((N-(3-(trifluoromethyl)phenyl)morpholine-4-carboxamido)methyl)benzoate

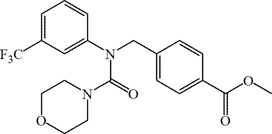 [SMILES: COC(=O)c1ccc(CN(C(=O)N2CCOCC2)c2cccc(C(F)(F)F)c2)cc1]

Methyl 4-((((4-nitrophenoxy)carbonyl)(3-(trifluoromethyl)phenyl)amino)methyl)benzoate (0.29 g, 0.60 mmol) was dissolved in dimethylformamide (10 ml), after which potassium carbonate (0.25 g, 1.81 mmol) and morpholine (0.05 mL, 0.60 mmol) were inserted. A resulting solution was subjected to reaction at 60° C. for two days, and then diluted with saturated ammonium chloride solution. An extraction was performed with ethyl acetate, after which a resulting extract was dried and filtered with anhydrous sodium sulfate, and then concentrated under reduced pressure. A residue was purified via column chromatography (silicon dioxide; ethyl acetate/hexane = 50%), such that a title compound (0.15 g, 60%) was obtained.

¹H NMR (400 MHz, DMSO-d₆) δ 7.97 (d, 2 H, J = 8.2 Hz), 7.43-7.32 (m, 5H), 7.20 (d, 1H, J = 8.0 Hz), 4.94 (s, 2H), 3.90 (s, 3H), 3.50 (t, 4 H, J = 4.8 Hz), 3.25 (t, 4 H, J = 4.8 Hz); MS (ESI) m/z 423 (M⁺ + H).

[Step 4] Synthesis of N-(4-(hydroxycarbamoyl)benzyl)-N-(3-(trifluoromethyl)phenyl)morpholine-4-carboxamide

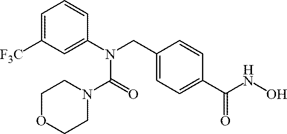 [SMILES: O=C(NO)c1ccc(CN(C(=O)N2CCOCC2)c2cccc(C(F)(F)F)c2)cc1]

Methyl 4-((N-(3-(trifluoromethyl)phenyl)morpholine-4-carboxamido)methyl)benzoate (0.15 g, 0.36 mmol) was dissolved in methanol (5 mL), after which hydroxylamine aqueous solution (50 wt%, 1 mL) and potassium hydroxide (0.10 g, 1.81 mmol) were inserted and stirred overnight. After a reaction was completed, a resulting solution was subjected to distillation under reduced pressure to remove methanol therefrom, after which an extraction was performed with ethyl acetate and water, such that work-up was done. A resulting extract was dried and filtered with anhydrous sodium sulfate, and then concentrated under reduced pressure. A residue was stirred in diethyl ether, after which a solid product was made, filtered and dried, such that a title compound (0.082 g, 54%) was obtained in a white solid form.

¹H NMR (400 MHz, MeOD-d₃) δ 11.14 (brs, 1H ), 8.99 (brs, 1 H), 7.85 (d, 2 H, J = 8.0 Hz), 7.66-7.27 (m, 6 H), 4.94 (s, 2 H), 3.41 (s, 2 H), 3.15 (s, 2 H). MS (ESI) m/z 424 (M⁺ + H).

### Preparation Example 2. Synthesis of N-(2,4-Difluorophenyl)-N-(4-(Hydroxycarbamoyl)Benzyl)Morpholine-4-Carboxamide [Compound 413]

[Step 1] Synthesis of methyl 4-((2,4-difluorophenylamino)methyl)benzoate

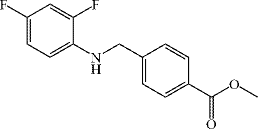 [SMILES: COC(=O)c1ccc(CNc2ccc(F)cc2F)cc1]

A 2,4-difluorobenzeneamine (3.0 g, 23.2 mmol) and methyl 4-formylbenzoate (3.81 g, 23.2 mmol) were dissolved in methanol (500 mL) and stirred at room temperature for two hours, after which acetic acid (1.33 mL, 23.2 mmol) and sodium cyanoborohydride (1.46 g, 23.2 mmol) were added and stirred for a day. Methanol was slightly removed by air, after which a solid was precipitated, filtered and dried, so that a title compound (2.9 g, 45%) was obtained in a white solid form.

[Step 2] Synthesis of methyl 4-(((2,4-difluorophenyl)((4-nitrophenoxy)carbonyl)amino)methyl)benzoate

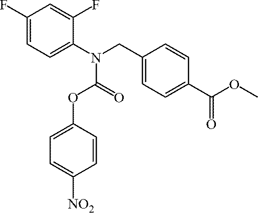 [SMILES: COC(=O)c1ccc(CN(C(=O)Oc2ccc([N+](=O)[O-])cc2)c2ccc(F)cc2F)cc1]

Methyl 4-((2,4-difluorophenylamido)methyl)benzoate (2 g, 7.21 mmol) and 4-nitrophenylchloroformate (1.45 g, 7.21 mmol) were dissolved in dichloromethane (50 mL) and stirred at room temperature for three days, after which water was added to extract an organic layer therefrom. The organic layer was washed with saturated sodium chloride aqueous solution, then dehydrated by anhydrous magnesium sulfate, and then concentrated under reduced pressure. A residue was dried, such that a title compound (2.5 g, 78%) was obtained in a yellow oil form.

[Step 3] Synthesis of methyl 4-((N-(2,4-difluorophenyl)morpholine-4-carboxamido)methyl)benzoate

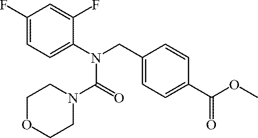 [SMILES: COC(=O)c1ccc(CN(C(=O)N2CCOCC2)c2ccc(F)cc2F)cc1]

Methyl 4-(((2,4-difluorophenyl)((4-nitrophenoxy)carbonyl)amino)methyl)benzoate (0.50 g, 1.13 mmol) and morpholine (0.098 mL, 1.13 mmol) were dissolved in dimethylformamide (10 mL) and heated and stirred at 60° C. for two days. Dimethylformamide was removed under reduced pressure, after which water was poured into the reaction mixture and an extraction was performed with ethyl acetate. An organic layer was washed with saturated sodium chloride aqueous solution, then dehydrated by anhydrous magnesium sulfate, and then concentrated under reduced pressure. A residue was purified via column chromatography (silicon dioxide; ethyl acetate/hexane = 30%) and concentrated, so that a title compound (0.44 g, 98%) was obtained in a colorless oil form.

[Step 4] Synthesis of N-(2,4-difluorophenyl)-N-(4-(hydroxycarbamoyl)benzyl)morpholine-4-carboxamide

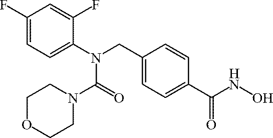 [SMILES: O=C(NO)c1ccc(CN(C(=O)N2CCOCC2)c2ccc(F)cc2F)cc1]

Methyl 4-((N-(2,4-difluorophenyl)morpholine-4-carboxamido)methyl)benzoate (0.10 g, 0.256 mmol) was dissolved in methanol (20 mL), after which hydroxylamine hydrochloride (0.089 g, 1.28 mmol) and potassium hydroxide (0.144 g, 2.56 mmol) were added and stirred, so that hydroxylamine (50 wt% aqueous solution; 0.329 mL, 5.12 mmol) was added dropwise and stirred at room temperature for three hours. After a reaction was completed, methanol was removed under reduced pressure, after which water was poured into the reaction mixture and an extraction was performed with ethyl acetate. An organic layer was washed with saturated sodium chloride aqueous solution, then dehydrated by anhydrous magnesium sulfate, and then concentrated under reduced pressure. After that, a resulting concentrate was dissolved in dichloromethane, after which hexane was added and a solid was precipitated, filtered and dried, so that a title compound (0.076 g, 76%) was obtained in a light yellow solid form.

¹H NMR (400 MHz, MeOD-d₃) δ 7.65 (d, 2 H, J = 8.3 Hz), 7.41 (d, 2 H, J = 8.2 Hz), 7.27 -7.25 (m, 1H), 7.04 - 6.96 (m, 2 H), 4.80 (s, 2 H), 3.46 - 3.43 (m, 4 H), 3.22 - 3.19 (m, 4 H); MS (ESI) m/z 392.1 (M⁺ + H).

### Preparation Example 3. Synthesis of N-(3-Chloro-4-Fluorophenyl)-N-(4-(Hydroxvcarbamoyl)Benzyl)Morpholine-4-Carboxamide [Compound 458]

[Step 1] Synthesis of methyl 4-((3-chloro-4-fluorophenylamino)methyl)benzoate

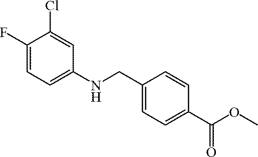 [SMILES: COC(=O)c1ccc(CNc2ccc(F)c(Cl)c2)cc1]

A 3-chloro-4-fluorobenzeneamine (2.0 g, 13.7 mmol) and methyl 4-formylbenzoate (2.26 g, 13.7 mmol) were dissolved in methanol (500 mL) and stirred at room temperature for three hours, after which acetic acid (0.786 mL, 13.7 mmol) and sodium cyanoborohydride (0.86 g, 13.7 mmol) were added and stirred for a day. Methanol was slightly removed by air, after which a solid was precipitated, filtered and dried, so that a title compound (2.9 g, 72%) was obtained in a gray solid form.

[Step 2] Synthesis of methyl 4-(((3-chloro-4-fluorophenyl)((4-nitrophenoxy)carbonyl)amino)methyl)benzoate

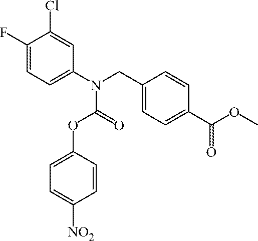 [SMILES: COC(=O)c1ccc(CN(C(=O)Oc2ccc([N+](=O)[O-])cc2)c2ccc(F)c(Cl)c2)cc1]

Methyl 4-((3-chloro-4-fluorophenylamido)methyl)benzoate (2.5 g, 8.51 mmol) and 4-nitrophenylchloroformate (2.06 g, 10.2 mmol) were dissolved in dichloromethane (50 mL) and stirred at room temperature for three days, after which water was added to extract an organic layer. The organic layer was washed with saturated sodium chloride aqueous solution, then dehydrated by anhydrous magnesium sulfate, and then concentrated under reduced pressure. A residue was dried, such that a title compound (2.5 g, 64%) was obtained in a violet oil form.

[Step 3] Synthesis of methyl 4-((N-(3-chloro-4-fluorophenyl)morpholine-4-carboxamido)methyl)benzoate

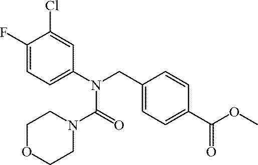 [SMILES: COC(=O)c1ccc(CN(C(=O)N2CCOCC2)c2ccc(F)c(Cl)c2)cc1]

Methyl 4-(((-3-chloro-4-fluorophenyl)((4-nitrophenoxy)carbonyl)amino)methyl)benzoate (0.20 g, 0.436 mmol) and morpholine (0.038 mL, 0.436 mmol) were dissolved in dimethylformamide (10 mL) and heated and stirred at 60° C. for 12 hours. Dimethylformamide was removed under reduced pressure, after which water was poured into the reaction mixture and an extraction was performed with ethyl acetate. The organic layer was washed with saturated sodium chloride aqueous solution, then dehydrated by anhydrous magnesium sulfate, and then concentrated under reduced pressure. A residue was purified via column chromatography (silicon dioxide; ethyl acetate/hexane = 20%) and concentrated, such that a title compound (0.022 g, 12%) was obtained in a colorless oil form.

[Step 4] Synthesis of N-(3-chloro-4-fluorophenyl)-N-(4-(hydroxycarbamoyl)benzyl)morpholine-4-carboxamide

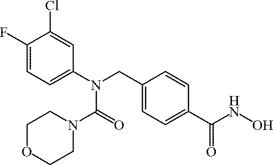 [SMILES: O=C(NO)c1ccc(CN(C(=O)N2CCOCC2)c2ccc(F)c(Cl)c2)cc1]

Methyl 4-((N-(3-chloro-4-fluorophenyl)morpholine-4-carboxamido)methyl)benzoate (0.050 g, 0.123 mmol) was dissolved in methanol (5 mL), after which hydroxylamine hydrochloride (0.043 g, 0.614 mmol) and potassium hydroxide (0.069 g, 1.23 mmol) were added and stirred, and then hydroxylamine (50 wt% aqueous solution; 0.158 mL, 2.46 mmol) was added dropwise and stirred at room temperature for three hours. After a reaction was completed, methanol was removed under reduced pressure, after which saturated sodium hydrogen carbonate aqueous solution was added, and then a solid was formed, filtered and dried, so that a title compound (0.017 g, 34%) was obtained in a white solid form.

¹H NMR (400 MHz, DMSO-d₆) δ 7.63 (d, 2 H, J = 8.2 Hz), 7.44 - 7.42 (m, 1H), 7.33 - 7.29 (m, 3 H), 7.15 - 7.11(m, 1 H), 4.84 (s, 2 H), 3.41 - 3.40 (m, 4 H), 3.14 - 3.12 (m, 4 H); MS (ESI) m/z 408.1 (M⁺+ H).

### Preparation Example 4. Synthesis of N-(4-(Hydroxycarbamoyl)Benzyl)-N-(3-Methoxyphenyl)Morpholine-4-Carboxamide [Compound 484]

[Step 1] Synthesis of methyl 4-(((3-methoxyphenyl)amino)methyl)benzoate

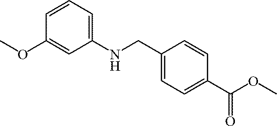

An m-anisidine (3.23 g, 26.2 mmol) and methyl 4-(bromomethyl)benzoate (5.00 g, 21.8 mmol) were dissolved in acetonitrile (50 mL), after which N,N-diisopropylethylamine (5.80 mL, 32.7 mmol) was added and stirred at room temperature for 16 hours. When a reaction was completed, an extraction was performed with ethyl acetate and saturated sodium hydrogen carbonate aqueous solution, after which an organic layer was dried by anhydrous magnesium sulfate and filtered. A remaining filtrate was concentrated under reduced pressure, after which a residue was purified via column chromatography (silicon dioxide; ethyl acetate/hexane = 5%) and concentrated, such that a title compound (5.14 g, 87%) was obtained in a bright yellow liquid form.

[Step 2] Synthesis of methyl 4-(((3-methoxyphenyl)((4-nitrophenoxy)carbonyl)amino)methyl)benzoate

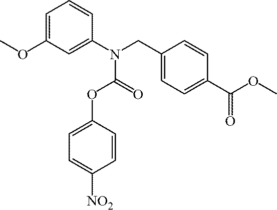

Methyl 4-(((3-methoxyphenyl)amino)methyl)benzoate (5.14 g, 18.9 mmol) and 4-nitrophenyl chloroformate (4.20 g, 20.8 mmol) were dissolved in acetonitrile (100 mL), after which potassium carbonate (3.93 g, 28.4 mmol) was added and stirred at room temperature for three hours. When a reaction was completed, an extraction was performed with ethyl acetate and saturated sodium hydrogen carbonate aqueous solution, after which an organic layer was dried by anhydrous magnesium sulfate and filtered. A remaining filtrate was concentrated under reduced pressure, after which a residue was purified via column chromatography (silicon dioxide; ethyl acetate/hexane = 20%) and concentrated, such that a title compound (5.88 g, 71%) was obtained in a yellow liquid form.

[Step 3] Synthesis of methyl 4-((N-(3-methoxyphenyl)morpholine-4-carboxamido)methyl)benzoate

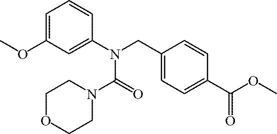

Methyl 4-(((3-methoxyphenyl)((4-nitrophenoxy)carbonyl)amino)methyl)benzoate (5.88 g, 13.5 mmol) was dissolved in dimethylformamide (50 mL), after which morpholine (2.35 g, 27.0 mmol) and potassium carbonate (5.60 g, 40.5 mmol) were added and stirred at 60° C. for 16 hours. When a reaction was completed, an extraction was performed with ethyl acetate and saturated ammonium chloride aqueous solution, after which an organic layer was dried by anhydrous magnesium sulfate and filtered. A remaining filtrate was concentrated under reduced pressure, after which a residue was purified via column chromatography (silicon dioxide; ethyl acetate/hexane = 30%) and concentrated, such that a title compound (3.69 g, 71%) was obtained in a yellow solid form.

[Step 4] Synthesis of N-(4-(hydroxycarbamoyl)benzyl)-N-(3-(methoxyphenyl)morpholine-4-carboxamide

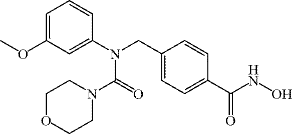 [SMILES: COc1cccc(N(Cc2ccc(C(=O)NO)cc2)C(=O)N2CCOCC2)c1]

Methyl 4-((N-(3-(methoxyphenyl)morpholine-4-carboxamido)methyl)benzoate (0.180 g, 0.468 mmol) was dissolved in methanol (10 mL), after which hydroxylamine (50.0 wt% aqueous solution; 1.43 mL, 23.4 mmol) was added at room temperature and then potassium hydroxide (0.263 g, 4.68 mmol) was added and stirred at the same temperature for 30 minutes. After that, a solvent was removed from the reaction mixture under reduced pressure. Saturated ammonium chloride aqueous solution was poured into the resulting concentrate, and an extraction was performed with ethyl acetate. An organic layer was washed with saturated sodium chloride aqueous solution, then dehydrated by anhydrous magnesium sulfate, and then concentrated under reduced pressure. A residue was crystallized at room temperature with dichloromethane (2 mL) and hexane (10 mL), such that a title compound (0.140 g, 78%) was obtained in a white solid form.

¹H NMR (400 MHz, DMSO-d₆) δ 7.63 (d, 2 H, J = 8.1 Hz), 7.32 (m, 2 H), 7.19 (t, 1 H, J = 8.4 Hz), 6.69 - 6.67 (m, 2 H), 6.62 (m, 1 H), 4.84 (s, 2 H), 3.69 (s, 3 H), 3.39 - 3.36 (m, 4 H), 3.15 - 3.12 (m, 4 H). MS (ESI) m/z 386 (M⁺ + H).

### Preparation Example 5. Synthesis of (N-(3-Fluorophenyl)-N-(4-Hydroxycarbamoyl)Benzyl)Morpholine-4-Carboxamide [Compound 530]

[Step 1] Synthesis of methyl 4-((3-fluorophenylamino)methyl)benzoate

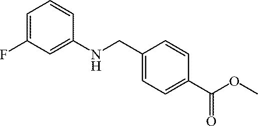 [SMILES: COC(=O)c1ccc(CNc2cccc(F)c2)cc1]

Methyl 4-formylbenzoate (1.47 g, 8.99 mmol) was dissolved in methanol (50 mL), after which 3-fluorobenzeneamine (1.0 g, 8.99 mmol) was inserted. A resulting solution was subjected to reaction at room temperature for three hours, after which sodium cyanoborohydride (NaCNBH₃, 0.56 g, 8.99 mmol) and acetic acid (1.03 mL, 17.99 mmol) were inserted. A reactant was subjected to reaction at room temperature for a day, after which a reaction solvent was removed under reduced pressure, then saturated sodium hydrogen carbonate aqueous solution was poured, and then an extraction was performed by ethyl acetate. An organic layer was dehydrated by anhydrous magnesium sulfate, and concentrated under reduced pressure. A residue was purified via column chromatography (silicon dioxide; ethyl acetate/hexane = 20%), such that a title compound (1.84 g, 79%) was obtained.

[Step 2] Synthesis of methyl 4-(((3-fluorophenyl)((4-nitrophenoxy)carbonyl)amino)methyl)benzoate

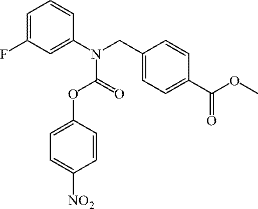 [SMILES: COC(=O)c1ccc(CN(C(=O)Oc2ccc([N+](=O)[O-])cc2)c2cccc(F)c2)cc1]

Methyl 4-((3-fluorophenylamino)methyl)benzoate (2.7 g, 10.4 mmol) and 4-nitrophenyl chloroformate (4.20 g, 20.8 mmol) were dissolved in acetonitrile (100 mL), after which potassium carbonate (4.32 g, 31.2 mmol) was inserted. A resulting solution was subjected to reaction at room temperature for a day and diluted with ethyl acetate. A reactant was washed with saturated sodium chloride aqueous solution, then dried and filtered with anhydrous sodium sulfate, and then concentrated under reduced pressure. A residue was purified via column chromatography (silicon dioxide; ethyl acetate/hexane = 20%), such that a title compound (2.65 g, 60%) was obtained in a colorless oil form.

[Step 3] Synthesis of methyl 4-((N-(3-fluorophenyl)morpholine-4-carboxamido)methyl)benzoate

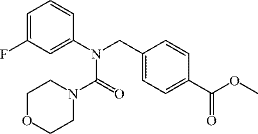 [SMILES: COC(=O)c1ccc(CN(C(=O)N2CCOCC2)c2cccc(F)c2)cc1]

Methyl 4-(((3-fluorophenyl)((4-nitrophenoxy)carbonyl)amino)methyl)benzoate ( 0.32 g, 0.75 mmol) was dissolved in dimethylformamide (5 ml), after which potassium carbonate (0.31 g, 2.24 mmol) and morpholine (0.13 mL, 1.49 mmol) were inserted. A resulting solution was subjected to reaction at 60° C. for a day, and then diluted with saturated ammonium chloride solution. An extraction was performed with ethyl acetate, after which a resulting extract was dried and filtered with anhydrous sodium sulfate, and then concentrated under reduced pressure. A residue was purified via column chromatography (silicon dioxide; ethyl acetate/hexane = 30%), such that a title compound (0.13 g, 45%) was obtained.

[Step 4] Synthesis of N-(3-fluorophenyl)-N-(4-hydroxycarbamoyl)benzyl)morpholine-4-carboxamide

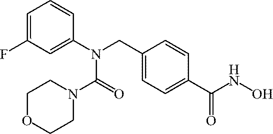 [SMILES: O=C(NO)c1ccc(CN(C(=O)N2CCOCC2)c2cccc(F)c2)cc1]

Methyl 4-((N-(3-fluorophenyl)morpholine-4-carboxamido)methyl)benzoate (0.108 g, 0.290 mmol) was dissolved in methanol (10 mL), after which hydroxylamine (50.0 wt% aqueous solution; 1.19 mL, 19.4 mmol) was added at room temperature, and then potassium hydroxide (0.156 g, 2.78 mmol) was added and stirred at the same temperature for 16 hours. Then, a solvent was removed from the reaction mixture under reduced pressure, after which saturated sodium hydrogen carbonate aqueous solution was poured into the resulting concentrate and an extraction was performed with ethyl acetate. The organic layer was washed with saturated sodium chloride aqueous solution, then dehydrated by anhydrous magnesium sulfate, and then concentrated under reduced pressure. A precipitate solid was filtered and dried, such that a title compound (0.062 g, 57%) was obtained in a white solid form.

¹H NMR (400 MHz, DMSO-d₆) δ 11.14 (brs, 1 H), 8.99 (brs, 1 H), 7.65 (d, 2 H, J = 7.0 Hz), 7.38-7.30 (m, 3H), 7.05-6.85 (m, 3H), 4.89 (s, 1H), 3.44-3.42 (m, 4H), 3.18-3.15 (m, 4H), 2.08 (s, 3H). MS (ESI) m/z 374 (M⁺ + H).

### Preparation Example 6. Synthesis of N-(3-Fluorophenyl)-N-(4-Hydroxycarbamoyl)Benzyl)-1,4-Oxazepane-4-Carboxamide [Compound 652]

[Step 1] Synthesis of methyl 4-((N-(3-fluorophenyl)-1,4-oxazepane-4-carboxamido)methyl)benzoate

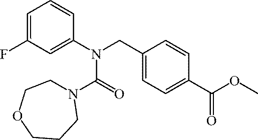 [SMILES: COC(=O)c1ccc(CN(C(=O)N2CCCOCC2)c2cccc(F)c2)cc1]

Methyl 4-(((3-fluorophenyl)((4-nitrophenoxy)carbonyl)amino)methyl)benzoate (0.290 g, 0.683 mmol) obtained in step 2 of Preparation Example 5, 1,4-oxazepane (0.188 g, 1.367 mmol) and potassium carbonate (0.283 g, 2.050 mmol) were dissolved in DMF (10 mL), after which the reaction solution was stirred at 60° C. for a day, then saturated NaHCO₃ aqueous solution was poured into the reaction mixture, and then an extraction was performed with ethyl acetate. An organic layer was washed with saturated sodium chloride aqueous solution, then dehydrated by anhydrous magnesium sulfate, and then concentrated under reduced pressure. A resulting concentrate was purified via column chromatography (silicon dioxide, 15 g cartridge; ethyl acetate/hexane = 20 to 50%) and concentrated, such that a title compound (0.116 g, 43.9%) was obtained in a colorless liquid form.

[Step 2] Synthesis of N-(3-fluorophenyl)-N-(4-hydroxycarbamoyl)benzyl)-1,4-oxazepane-4-carboxamide

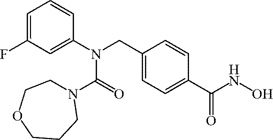 [SMILES: O=C(NO)c1ccc(CN(C(=O)N2CCCOCC2)c2cccc(F)c2)cc1]

Methyl 4-((N-(3-(fluorophenyl)-1,4-oxazepane-4-carboxamide)methyl)benzoate (0.116 g, 0.3 mmol) was dissolved in methanol (10 mL), after which hydroxylamine aqueous solution (50 wt%, 1 mL) and potassium hydroxide (0.168 g, 3.01 mmol) were inserted and stirred overnight. After a reaction was completed, a resulting solution was subjected to distillation under reduced pressure to remove methanol therefrom, after which an extraction was performed with ethyl acetate and water, such that work-up was done. A resulting extract was dried and filtered with anhydrous sodium sulfate and concentrated under reduced pressure, after which a residue was stirred in diethyl ether, such that a solid product was made, filtered and dried, and thus a title compound (0.032 g, 27.5%) was obtained in a white solid form.

### Example 1 Analysis of Therapeutic Effect in Animal Model With Induced COPD

### Example 1-1 Preparation of Animal Model With Induced COPD (Smoking + Poly I:C COPD Model)

A C57BL/6 mouse (female, seven weeks old) was placed in a sealed smoking box and exposed to cigarette smoke for a certain period of time to induce COPD disease. Specifically, the mouse was put into the smoking box, after which a cigarette was lit and its filter was connected to a tube under a negative pressure, so that cigarette smoke could be continuously produced. Then, the smoking box was blocked, so that the mouse was forced to inhale the cigarette smoke while breathing in. The mouse was allowed to smoke for a total of four weeks. The mouse was exposed to one, two and four cigarettes once on the first, second and third days of the first week, respectively, and again exposed to four cigarettes twice and three times on the fourth and fifth days, respectively. After that, the mouse was exposed to four cigarettes three times a day for the remaining three weeks. Poly I:C (polyinosinic:polycytidylic acid) was administered into a nasal cavity of the anesthetized mouse twice a week in the third and fourth weeks during induction of smoking, so that a model of smoking + Poly I:C COPD (chronic obstructive pulmonary disease) was induced.

In order to confirm a drug administration effect of the compound according to the present invention, mice with induced COPD were divided into each group of six mice, and each of the groups was is classified as shown in a following table 1 according to an administered substance [vehicle (Veh), compound 374 (SM+1), or compound 458 (SM+2)], a route of administration [oral administration (P.O.)], and an administration interval [daily (Q.D.)].

TABLE 1

| Group | Administered dose (mg/kg) | Administrati on route | Administrati on interval | Number of animals |
|---|---|---|---|---|
| Veh | - | P.O, | Q.D. | 6 |
| SM+1 | 30 | P.O, | Q.D. | 6 |
| SM+2 | 30 | P.O, | Q.D. | 6 |

(Veh: 0.5% MC in DW)

### Example 1-2 Evaluation of Immune Cell Infiltration

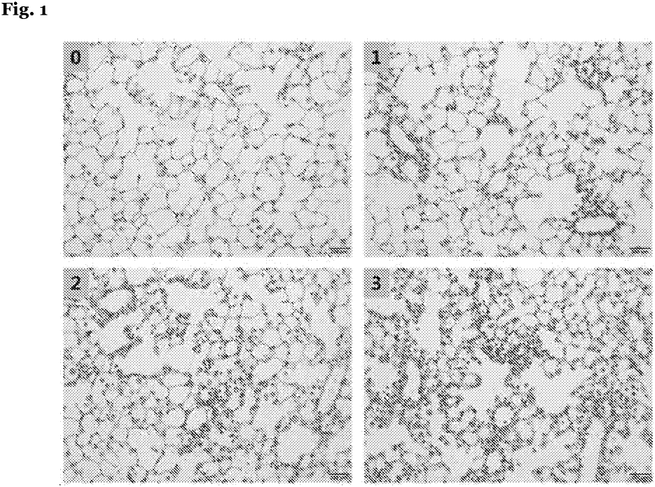

In order to confirm an effect of the compound according to the present invention on preventing or treating chronic obstructive pulmonary disease, a degree of immune cell infiltration was evaluated. After lungs were removed from the mice of each group, sections of lung tissues were stained with H&E and subjected to pathological analysis. According to a reference image of FIG. 1, a degree of cellular infiltration was determined as a score of 0 to 3, and the score was measured by observing slides for each group [FIG. 1: 0-none, 1-mild, 2-moderate, and 3-severe].

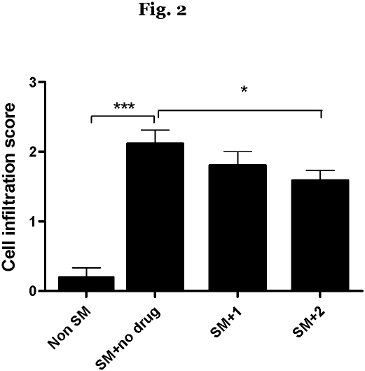

As a result, as shown in FIG. 2, a group dosed with the compound according to the present invention (SM+1 and SM+2) showed a decrease in cellular infiltration scores compared to a group of smoking + Poly I:C (SM+no drug). Thus, it could be understood that the compound according to the present invention shows an effect of inhibiting the infiltration of immune cells, and thus is useful in preventing or treating chronic obstructive pulmonary disease.

### Example 1-3 Analysis of Bronchoalveolar Lavage Fluid (BAL Fluid)

In order to confirm an effect of the compound according to the present invention on preventing or treating chronic obstructive pulmonary disease, bronchoalveolar lavage fluid (BAL fluid) was analyzed. The 2 mL of the BAL fluid was separated from the mice of each group and centrifuged. After that, the resulting fluid was suspended in a phosphate buffer solution (PBS), after which the number of total cells was counted, and 2 × 10⁵ cells were attached to a slide through a cytospin. After staining with a diff-quick stain kit, the resulting cells were classified into macrophage, neutrophil, etc. based on the properties of the stained cells, so as to count a total of 300 cells, after which a rate of respective cells was calculated with regard to the number of total cells to make a comparison among the groups.

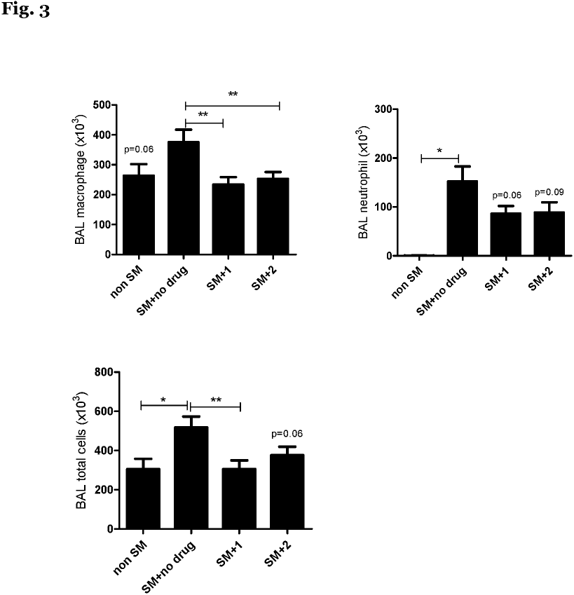

As a result, as shown in FIG. 3, the group dosed with the compound according to the present invention (SM+1 and SM+2) was compared with the group of smoking + Poly I:C (SM+no drug), and thus the number of total cells, macrophages and neutrophils was decreased. Thus, it could be understood that the compound according to the present invention shows an effect of inhibiting the infiltration of inflammatory cells, and thus is useful in preventing or treating chronic obstructive pulmonary disease. (* P < 0.05, ** P < 0.01, and *** P < 0.001)

### Example 1-4 Analysis of Expression of Inflammatory Cytokines in Lung Tissue

In order to confirm an effect of the compound according to the present invention on preventing or treating chronic obstructive pulmonary disease, an expression of inflammatory cytokines in lung tissues was analyzed. Lungs were removed from mice of each group, after which RNA was isolated from the lung tissues. For each RNA, cDNA synthesis was performed by using a reverse transcriptase, after which an expression of inflammatory cytokine markers such as IL-6, IFN-y, MCP-1, and TNF-α was analyzed through real-time PCR, so as to compare a degree of induced inflammation among the groups. In this case, a sequence of a primer used in the experiment is the same as shown in a following table 2. (* P < 0.05, ** P < 0.01, and *** P < 0.001)

TABLE 2

| Gene | Sequence | | SEQ ID NO: |
|---|---|---|---|
| mIL-6 | Forward | CTGCAAGAGACTTCCATCCAG | 1 |
| | Reverse | AGTGGTATAGACAGGTCTGTT GG | 2 |
| mIFN-γ | Forward | ATGAACGCTACACACTGCATC | 3 |
| | Reverse | CCATCCTTTTGCCAGTTCCTC | 4 |
| mMCP-1 | Forward | TTAAAAACCTGGATCGGAAC CAA | 5 |
| | Reverse | GCATTAGCTTCAGATTTACGG GT | 6 |
| mTNF-α | Forward | CTGAACTTCGGGGTGATCGG | 7 |
| | Reverse | GGCTTGTCACTCGAATTTTGA GA | 8 |

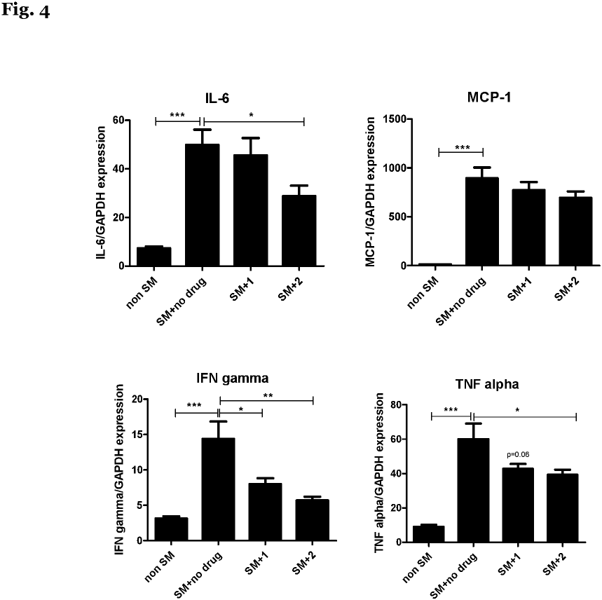

As a result, as shown in FIG. 4, the group dosed with the compound according to the present invention (SM+1 and SM+2) was compared with the group of smoking + Poly I:C (SM+no drug), and thus the expression of IL-6, IFN-y, MCP-1 and TNF-α was decreased. Thus, it could be understood that the compound according to the present invention shows an antiinflammatory effect, and thus is useful in preventing or treating chronic obstructive pulmonary disease.

### Example 2 Inhibitory Effect on TGF-β1-Induced EMT

In order to confirm an effect of the compound according to the present invention on preventing or treating chronic obstructive pulmonary disease, an expression of an epithelial marker protein (E-Cad) and a mesenchymal marker protein (N-Cad) was analyzed. A549 cells were cultured in RPMI-1640 medium containing 10% fetal bovine serum (FBS) and 1% penicillin-streptomycin (P/S). The 1.5×10⁵ cells were plated in a 6-well plate and incubated overnight. The cells were cultured with 5 ng/mL of recombinant human transforming growth factor beta 1 (TGF-β1) to induce an epithelial-to-mesenchymal transition (EMT). TGF-β1 was diluted with RPMI-1640 (1% FBS, 1% P/S) and incubated with the cells for 48 hours. The 500 µL of culture medium was loaded on a centrifugal filter (Amicon® Ultra-0.5 mL) and centrifuged at 14,000×g for 10 minutes. The enriched culture medium was boiled with 15 µL of 4X sample buffer at 100° C. for five minutes and analyzed by using the Western blot. The cells were lysed by using an RIPA buffer containing a protease inhibitor cocktail and a phosphatase inhibitor cocktail. A lysate was incubated on ice for three minutes and centrifuged at 13,000×g, 4° C. for 20 minutes. A supernatant was quantified by using a BCA protein assay kit and analyzed by the Western blot. The prepared sample was loaded on NuPAGE Novex 4-12% Bis-Tris gel and decomposed at 120 V Proteins were transferred to a nitrocellulose membrane by using an iBlot machine. The membrane was blocked by using EzBlock Chemi according to the manufacturer’s instructions. Antibodies such as E-cadherin (E-Cad), N-cadherin (N-cad), Fibronectin-EDA (FN-EDA), etc., were used. After reaction with a horseradish peroxidase (HRP)-conjugated secondary antibody, immunoreactive proteins were visualized by using Amersham® ECL select® Western blot detection reagent, and detected by ChemiDoc® MP imaging system. The intensity of the band was quantified with ImageJ software and normalized to β-actin.

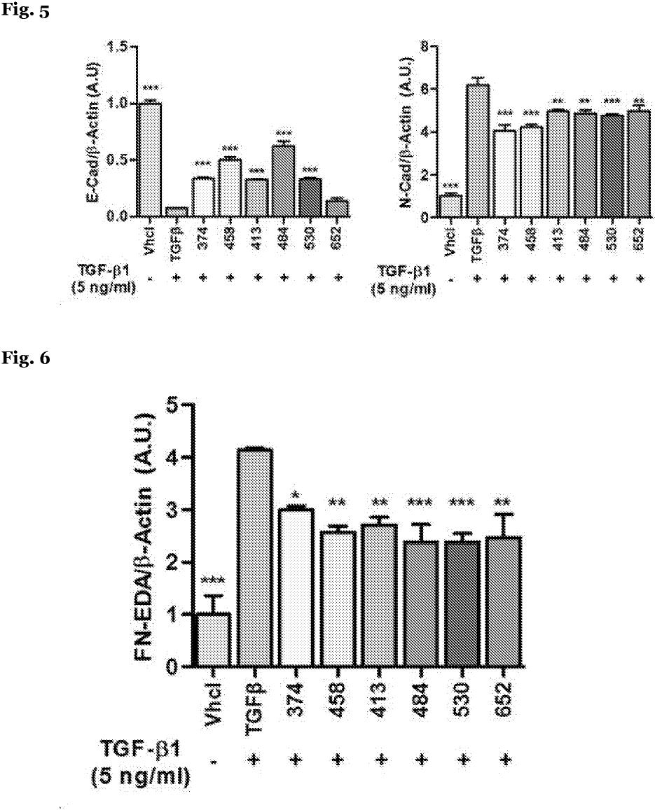

In result, as shown in FIG. 5, the groups treated with the compound according to the present invention (374, 458, 413, 484, 530 and 652) showed a remarkable increase in an expression of E-Cad, which is an epithelial marker protein, and a remarkable decrease in an expression of N-Cad, which is a mesenchymal marker protein, compared to Vhcl (TGF-β1).

Thus, it could be understood that the compound according to the present invention shows an effect of inhibiting TGF-B1-induced EMT, and thus is useful in preventing or treating chronic obstructive pulmonary disease. (* P < 0.05, ** P < 0.01, and *** P < 0.001)

### Example 3 Effect on FN-EDA Protein Reduction

In order to confirm an effect of the compound according to the present invention on preventing or treating chronic obstructive pulmonary disease, an expression of FN-EDA, a key extracellular matrix (ECM) protein, was analyzed. The analysis was performed by the same method as described in Example 2 above.

In result, as shown in FIG. 6, the groups treated with the compound according to the present invention (374, 458, 413, 484, 530 and 652) showed a remarkable decrease in an expression of a key ECM protein FN-EDA compared to Vhcl (TGF-β1).

Thus, it could be understood that the compound according to the present invention shows an effect of reducing the key ECM protein FN-EDA, and thus is useful in preventing or treating chronic obstructive pulmonary disease. (* P < 0.05, ** P < 0.01, and *** P < 0.001)

### Example 4 Statistical Analysis

Graph Pad Prism 5.0 software (post hoc: Dunnett) was used to perform unpaired T-test, and all data were expressed as mean±SEM. P < 0.05 was considered to be statistically significant.

While specific portions of the present invention have been described in detail above, it is apparent to those skilled in the art that such detailed descriptions are set forth to illustrate exemplary embodiments only, but are not construed to limit the scope of the present invention. Thus, it should be understood that the substantial scope of the present invention is defined by the accompanying claims and equivalents thereto.

1. A pharmaceutical composition for preventing or treating chronic obstructive pulmonary disease, comprising a compound represented by a following chemical formula I, optical isomers thereof or pharmaceutically acceptable salts thereof as an effective ingredient: 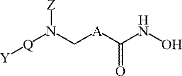
wherein
A is 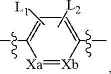
Xa and Xb are each independently CH or N,
L₁ and L₂ are each independently hydrogen, halogen, —CF₃, or -C₁₋₃ straight or branched chain alkyl,
Q is C(=O), S(=O)₂, S(=O) or C(=NH), and
Y is selected from a following group: 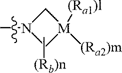 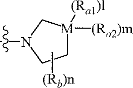 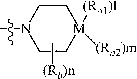 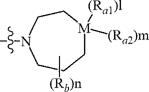 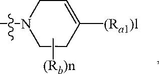
M is C, N, O, S or S(═O)₂ (at this time, when M is C, 1 and m are ₁; when M is N, l is 1 and m is o; and when M is O, S or S(=O)₂, l and m are o),
Rₐ₁ and Rₐ₂ are each independently hydrogen; hydroxy; -C₁₋₄ straight or branched chain alkyl, which is unsubstituted or substituted with at least one halogen; -C₁₋₄ straight or branched chain alcohol; benzhydryl; -C₁₋₄ straight or branched chain alkyl, which is substituted with a saturated or unsaturated five to seven-membered heterocyclic compound having one to three heteroatoms out of N, O or S as a ring member (at this time, the heterocyclic compound may be unsubstituted or at least one hydrogen may be optionally substituted with OH, OCH₃, CH₃, CH₂CH₃ or halogen); a saturated or unsaturated five to seven-membered heterocyclic compound having one to three heteroatoms out of N, O or S as a ring member (at this time, the heterocyclic compound may be unsubstituted or at least one hydrogen may be optionally substituted with OH, OCH₃, CH₃, CH₂CH₃ or halogen); phenyl, which is unsubstituted or in which at least one hydrogen is substituted with halogen, C₁₋₄ alkoxy, C₁₋₂ alkyl or hydroxy; benzyl, which is unsubstituted or in which at least one hydrogen is substituted with halogen, C₁₋₄ alkoxy, C₁₋₂ alkyl or hydroxy; —S(═O)₂CH₃; halogen; -C₁₋ 6 straight or branched chain alkoxy; -C₂₋₆ alkoxyalkyl; —C(═O)Rₓ, in which Rx is C₁₋₃
straight or branched chain alkyl or C₃-₁₀ cycloalkyl; 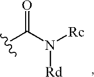
in which R_{c} and R_{d}
are independently hydrogen or C₁₋₃ straight or branched chain alkyl; 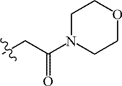
or 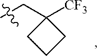
n is an integer of 0, 1 or 2,
R_{b} is hydrogen; hydroxy; -C₁₋₆ straight or branched chain alkyl, which is unsubstituted or in which at least one hydrogen is substituted with halogen; —C(═O)CH₃; -C₁₋₄ straight or branched chain hydroxyalkyl; -C₁₋₆ straight or branched chain alkoxy; -C₂₋₆ straight or branched chain alkoxyalkyl; —CF₃; halogen; or 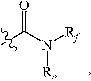
Rₑ and R_{f} are each independently hydrogen or -C₁₋₃ straight or branched chain alkyl, and
Z is selected from a following group: 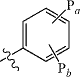 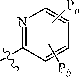 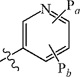 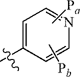 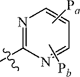 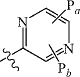 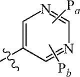 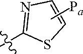 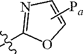 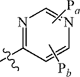 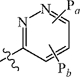 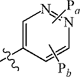
Pₐ and P_{b} are each independently 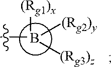
hydrogen; hydroxy; -C₁₋₄ straight or branched chain alkyl, which is unsubstituted or in which at least one hydrogen is substituted with halogen; halogen; —CF₃; —OCF₃; —CN; -C₁₋₆ straight or branched chain alkoxy; -C₂₋₆ straight or branched chain alkyl alkoxy; —CH₂F; or -C₁₋₃ alcohol,
in which 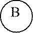
is a ring selected from phenyl, pyridine, pyrimidine, thiazole, indole, indazole, piperazine, quinoline, furan, tetrahydropyridine, piperidine or a following group: 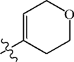 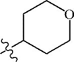 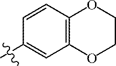 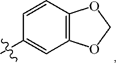
x, y and z are each independently an integer of o or ₁, and
R_{g1}, R_{g2} and R_{g3} are each independently selected from hydrogen; hydroxy; -C₁₋₃ alkyl; —CF₃ ; -C₁₋₆ straight or branched chain alkoxy; -C₂₋₆ straight or branched chain alkyl alkoxy; —C(═O)CH₃ ; -C₁₋₄ straight or branched chain hydroxyalkyl; —N(CH₃)₂ ; halogen; phenyl; —S((═O)₂)CH₃ ; or a following group: 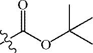 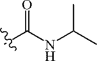 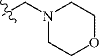 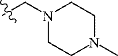 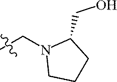 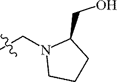 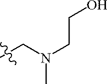 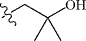 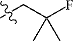 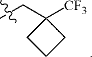
.

2. The pharmaceutical composition according to claim 1, wherein the compound represented by the above chemical formula I is a compound represented by a following chemical formula Ia: 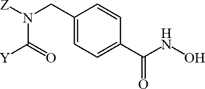
wherein
Y is selected from a following group: 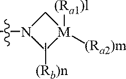 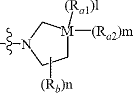 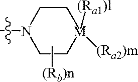 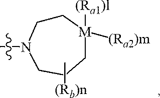
in which M, 1, m, n, Rₐ₁, Rₐ₂, and R_{b} are the same as defined in claim 1, respectively,
Z is 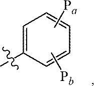
Pₐ and P_{b} are each independently hydrogen; hydroxy; -C₁₋₄ straight or branched chain alkyl, which is unsubstituted or in which at least one hydrogen is substituted with halogen; halogen; —CF₃; —OCF₃; —CN; -C₁₋₆ straight or branched chain alkoxy; -C₂₋₆ straight or branched chain alkyl alkoxy; —CH₂F; or -C₁₋₃ alcohol.

3. The pharmaceutical composition according to claim 2, wherein Y is selected from a following group: 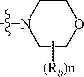 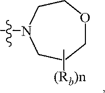
in which n and R_{b} are the same as defined in claim 1, respectively,
Z is 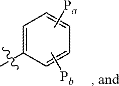
Pₐ and P_{b} are each independently hydrogen; halogen; —CF₃; or -C₁₋₆ straight or branched chain alkoxy.

4. The pharmaceutical composition according to claim 1, wherein the compound represented by the above chemical formula I is a compound described in a following table:

| Compou nd | Structure | Compo und | Structure |
|---|---|---|---|
| 252 | 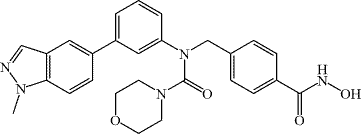 [SMILES: Cn1ncc2cc(-c3cccc(N(Cc4ccc(C(=O)NO)cc4)C(=O)N4CCOCC4)c3)ccc21] | 262 | 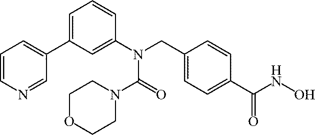 [SMILES: O=C(NO)c1ccc(CN(C(=O)N2CCOCC2)c2cccc(-c3cccnc3)c2)cc1] |
| 253 | 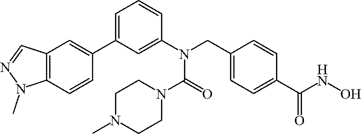 [SMILES: CN1CCN(C(=O)N(Cc2ccc(C(=O)NO)cc2)c2cccc(-c3ccc4c(cnn4C)c3)c2)CC1] | 263 | 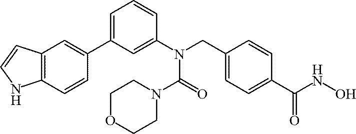 [SMILES: O=C(NO)c1ccc(CN(C(=O)N2CCOCC2)c2cccc(-c3ccc4[nH]ccc4c3)c2)cc1] |
| 254 | 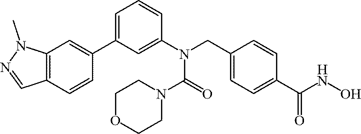 [SMILES: Cn1ncc2ccc(-c3cccc(N(Cc4ccc(C(=O)NO)cc4)C(=O)N4CCOCC4)c3)cc21] | 279 | 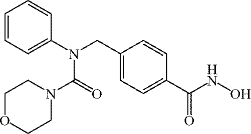 [SMILES: O=C(NO)c1ccc(CN(C(=O)N2CCOCC2)c2ccccc2)cc1] |
| 255 | 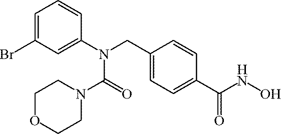 [SMILES: O=C(NO)c1ccc(CN(C(=O)N2CCOCC2)c2cccc(Br)c2)cc1] | 280 | 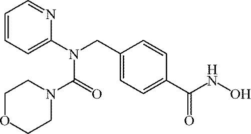 [SMILES: O=C(NO)c1ccc(CN(C(=O)N2CCOCC2)c2ccccn2)cc1] |
| 256 | 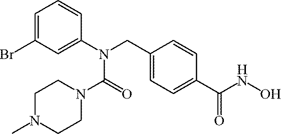 [SMILES: CN1CCN(C(=O)N(Cc2ccc(C(=O)NO)cc2)c2cccc(Br)c2)CC1] | 281 | 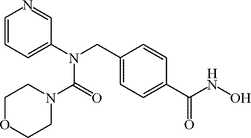 [SMILES: O=C(NO)c1ccc(CN(C(=O)N2CCOCC2)c2cccnc2)cc1] |
| 260 | 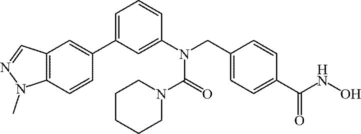 [SMILES: Cn1ncc2cc(-c3cccc(N(Cc4ccc(C(=O)NO)cc4)C(=O)N4CCCCC4)c3)ccc21] | 309 | 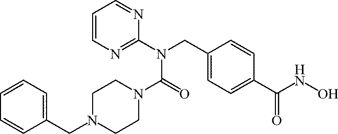 [SMILES: O=C(NO)c1ccc(CN(C(=O)N2CCN(Cc3ccccc3)CC2)c2ncccn2)cc1] |
| 261 | 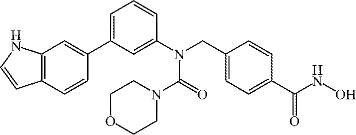 [SMILES: O=C(NO)c1ccc(CN(C(=O)N2CCOCC2)c2cccc(-c3ccc4cc[nH]c4c3)c2)cc1] | 311 | 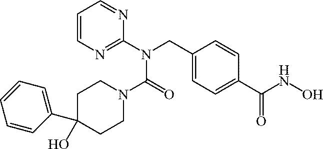 [SMILES: O=C(NO)c1ccc(CN(C(=O)N2CCC(O)(c3ccccc3)CC2)c2ncccn2)cc1] |
| 312 | 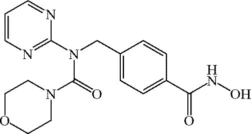 [SMILES: O=C(NO)c1ccc(CN(C(=O)N2CCOCC2)c2ncccn2)cc1] | 334 | 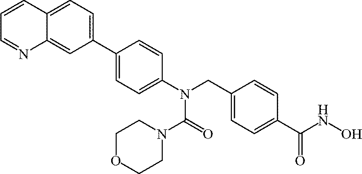 [SMILES: O=C(NO)c1ccc(CN(C(=O)N2CCOCC2)c2ccc(-c3ccc4cccnc4c3)cc2)cc1] |
| 313 | 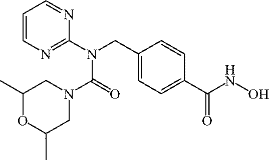 [SMILES: CC1CN(C(=O)N(Cc2ccc(C(=O)NO)cc2)c2ncccn2)CC(C)O1] | 335 | 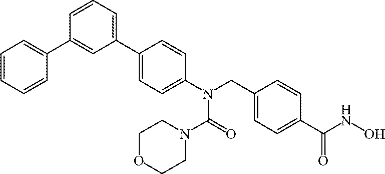 [SMILES: O=C(NO)c1ccc(CN(C(=O)N2CCOCC2)c2ccc(-c3cccc(-c4ccccc4)c3)cc2)cc1] |
| 329 | 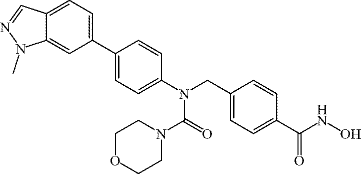 [SMILES: Cn1ncc2ccc(-c3ccc(N(Cc4ccc(C(=O)NO)cc4)C(=O)N4CCOCC4)cc3)cc21] | 336 | 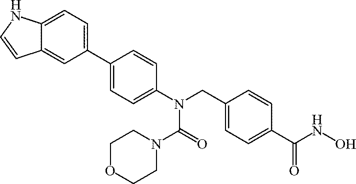 [SMILES: O=C(NO)c1ccc(CN(C(=O)N2CCOCC2)c2ccc(-c3ccc4[nH]ccc4c3)cc2)cc1] |
| 330 | 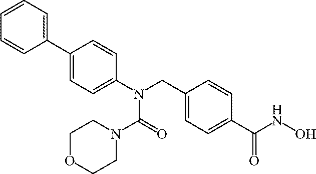 [SMILES: O=C(NO)c1ccc(CN(C(=O)N2CCOCC2)c2ccc(-c3ccccc3)cc2)cc1] | 337 | 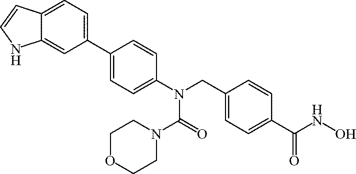 [SMILES: O=C(NO)c1ccc(CN(C(=O)N2CCOCC2)c2ccc(-c3ccc4cc[nH]c4c3)cc2)cc1] |
| 331 | 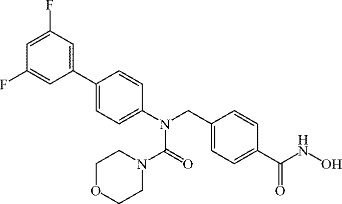 [SMILES: O=C(NO)c1ccc(CN(C(=O)N2CCOCC2)c2ccc(-c3cc(F)cc(F)c3)cc2)cc1] | 338 | 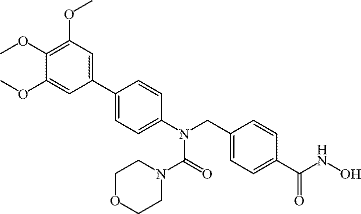 [SMILES: COc1cc(-c2ccc(N(Cc3ccc(C(=O)NO)cc3)C(=O)N3CCOCC3)cc2)cc(OC)c1OC] |
| 332 | 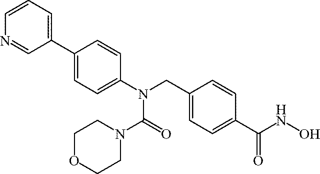 [SMILES: O=C(NO)c1ccc(CN(C(=O)N2CCOCC2)c2ccc(-c3cccnc3)cc2)cc1] | 339 | 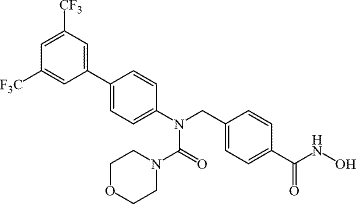 [SMILES: O=C(NO)c1ccc(CN(C(=O)N2CCOCC2)c2ccc(-c3cc(C(F)(F)F)cc(C(F)(F)F)c3)cc2)cc1] |
| 333 | 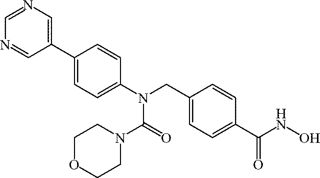 [SMILES: O=C(NO)c1ccc(CN(C(=O)N2CCOCC2)c2ccc(-c3cncnc3)cc2)cc1] | 340 | 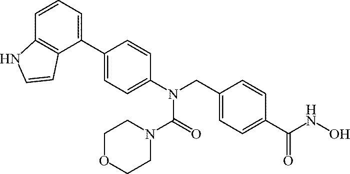 [SMILES: O=C(NO)c1ccc(CN(C(=O)N2CCOCC2)c2ccc(-c3cccc4[nH]ccc34)cc2)cc1] |
| 341 | 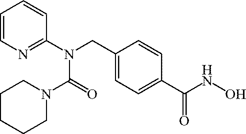 [SMILES: O=C(NO)c1ccc(CN(C(=O)N2CCCCC2)c2ccccn2)cc1] | 356 | 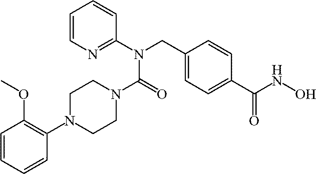 [SMILES: COc1ccccc1N1CCN(C(=O)N(Cc2ccc(C(=O)NO)cc2)c2ccccn2)CC1] |
| 342 | 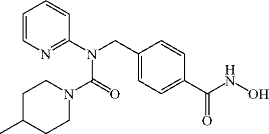 [SMILES: CC1CCN(C(=O)N(Cc2ccc(C(=O)NO)cc2)c2ccccn2)CC1] | 357 | 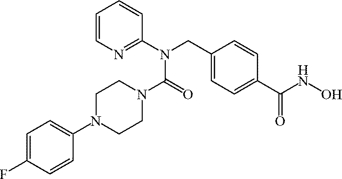 [SMILES: O=C(NO)c1ccc(CN(C(=O)N2CCN(c3ccc(F)cc3)CC2)c2ccccn2)cc1] |
| 343 | 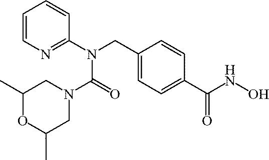 [SMILES: CC1CN(C(=O)N(Cc2ccc(C(=O)NO)cc2)c2ccccn2)CC(C)O1] | 358 | 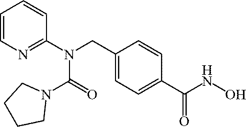 [SMILES: O=C(NO)c1ccc(CN(C(=O)N2CCCC2)c2ccccn2)cc1] |
| 352 | 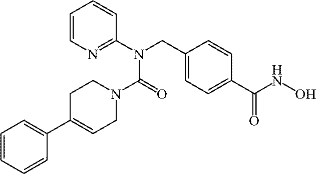 [SMILES: O=C(NO)c1ccc(CN(C(=O)N2CC=C(c3ccccc3)CC2)c2ccccn2)cc1] | 370 | 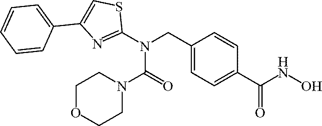 [SMILES: O=C(NO)c1ccc(CN(C(=O)N2CCOCC2)c2nc(-c3ccccc3)cs2)cc1] |
| 353 | 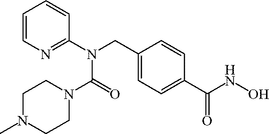 [SMILES: CN1CCN(C(=O)N(Cc2ccc(C(=O)NO)cc2)c2ccccn2)CC1] | 371 | 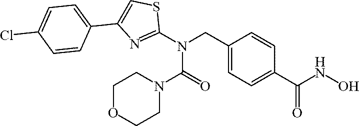 [SMILES: O=C(NO)c1ccc(CN(C(=O)N2CCOCC2)c2nc(-c3ccc(Cl)cc3)cs2)cc1] |
| 354 | 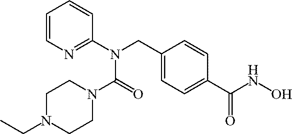 [SMILES: CCN1CCN(C(=O)N(Cc2ccc(C(=O)NO)cc2)c2ccccn2)CC1] | 372 | 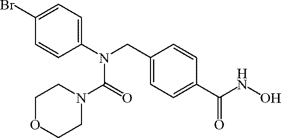 [SMILES: O=C(NO)c1ccc(CN(C(=O)N2CCOCC2)c2ccc(Br)cc2)cc1] |
| 355 | 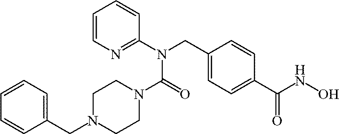 [SMILES: O=C(NO)c1ccc(CN(C(=O)N2CCN(Cc3ccccc3)CC2)c2ccccn2)cc1] | 374 | 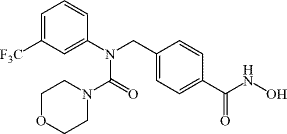 [SMILES: O=C(NO)c1ccc(CN(C(=O)N2CCOCC2)c2cccc(C(F)(F)F)c2)cc1] |
| 376 | 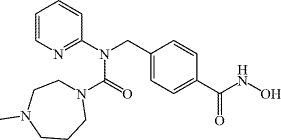 [SMILES: CN1CCCN(C(=O)N(Cc2ccc(C(=O)NO)cc2)c2ccccn2)CC1] | 385 | 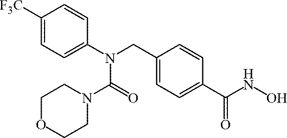 [SMILES: O=C(NO)c1ccc(CN(C(=O)N2CCOCC2)c2ccc(C(F)(F)F)cc2)cc1] |
| 377 | 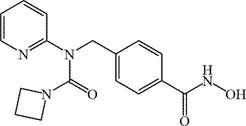 [SMILES: O=C(NO)c1ccc(CN(C(=O)N2CCC2)c2ccccn2)cc1] | 386 | 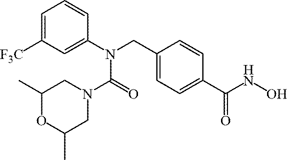 [SMILES: CC1CN(C(=O)N(Cc2ccc(C(=O)NO)cc2)c2cccc(C(F)(F)F)c2)CC(C)O1] |
| 379 | 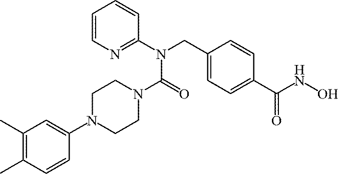 [SMILES: Cc1ccc(N2CCN(C(=O)N(Cc3ccc(C(=O)NO)cc3)c3ccccn3)CC2)cc1C] | 389 | 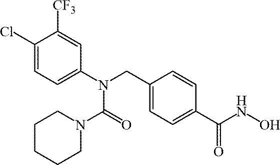 [SMILES: O=C(NO)c1ccc(CN(C(=O)N2CCCCC2)c2ccc(Cl)c(C(F)(F)F)c2)cc1] |
| 380 | 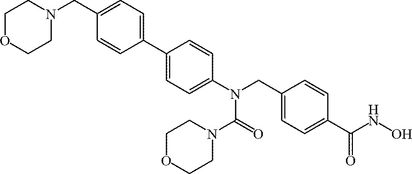 [SMILES: O=C(NO)c1ccc(CN(C(=O)N2CCOCC2)c2ccc(-c3ccc(CN4CCOCC4)cc3)cc2)cc1] | 390 | 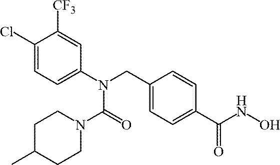 [SMILES: CC1CCN(C(=O)N(Cc2ccc(C(=O)NO)cc2)c2ccc(Cl)c(C(F)(F)F)c2)CC1] |
| 381 | 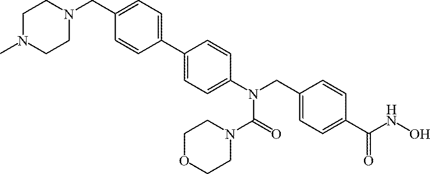 [SMILES: CN1CCN(Cc2ccc(-c3ccc(N(Cc4ccc(C(=O)NO)cc4)C(=O)N4CCOCC4)cc3)cc2)CC1] | 391 | 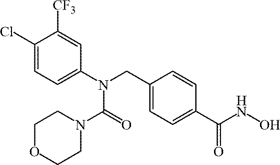 [SMILES: O=C(NO)c1ccc(CN(C(=O)N2CCOCC2)c2ccc(Cl)c(C(F)(F)F)c2)cc1] |
| 382 | 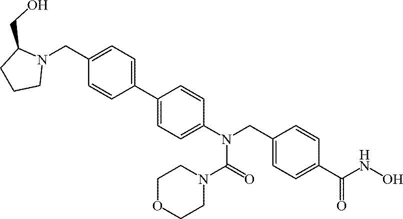 [SMILES: O=C(NO)c1ccc(CN(C(=O)N2CCOCC2)c2ccc(-c3ccc(CN4CCC[C@H]4CO)cc3)cc2)cc1] | 392 | 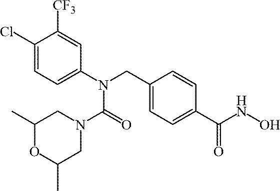 [SMILES: CC1CN(C(=O)N(Cc2ccc(C(=O)NO)cc2)c2ccc(Cl)c(C(F)(F)F)c2)CC(C)O1] |
| 383 | 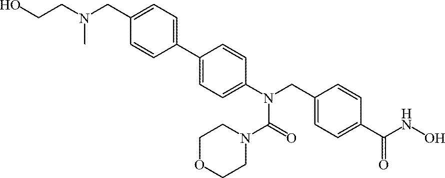 [SMILES: CN(CCO)Cc1ccc(-c2ccc(N(Cc3ccc(C(=O)NO)cc3)C(=O)N3CCOCC3)cc2)cc1] | 393 | 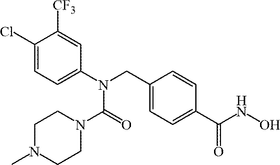 [SMILES: CN1CCN(C(=O)N(Cc2ccc(C(=O)NO)cc2)c2ccc(Cl)c(C(F)(F)F)c2)CC1] |
| 394 | 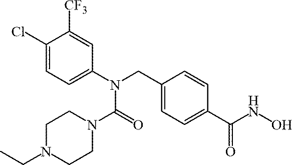 [SMILES: CCN1CCN(C(=O)N(Cc2ccc(C(=O)NO)cc2)c2ccc(Cl)c(C(F)(F)F)c2)CC1] | 401 | 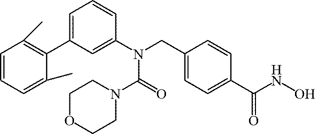 [SMILES: Cc1cccc(C)c1-c1cccc(N(Cc2ccc(C(=O)NO)cc2)C(=O)N2CCOCC2)c1] |
| 395 | 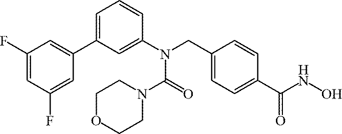 [SMILES: O=C(NO)c1ccc(CN(C(=O)N2CCOCC2)c2cccc(-c3cc(F)cc(F)c3)c2)cc1] | 402 | 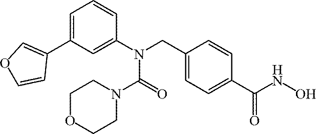 [SMILES: O=C(NO)c1ccc(CN(C(=O)N2CCOCC2)c2cccc(-c3ccoc3)c2)cc1] |
| 396 | 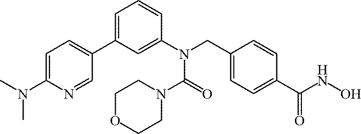 [SMILES: CN(C)c1ccc(-c2cccc(N(Cc3ccc(C(=O)NO)cc3)C(=O)N3CCOCC3)c2)cn1] | 403 | 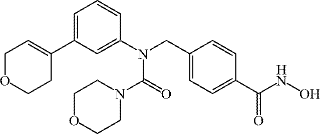 [SMILES: O=C(NO)c1ccc(CN(C(=O)N2CCOCC2)c2cccc(C3=CCOCC3)c2)cc1] |
| 397 | 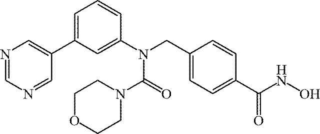 [SMILES: O=C(NO)c1ccc(CN(C(=O)N2CCOCC2)c2cccc(-c3cncnc3)c2)cc1] | 404 | 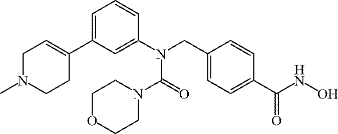 [SMILES: CN1CC=C(c2cccc(N(Cc3ccc(C(=O)NO)cc3)C(=O)N3CCOCC3)c2)CC1] |
| 398 | 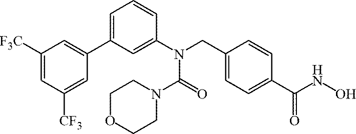 [SMILES: O=C(NO)c1ccc(CN(C(=O)N2CCOCC2)c2cccc(-c3cc(C(F)(F)F)cc(C(F)(F)F)c3)c2)cc1] | 405 | 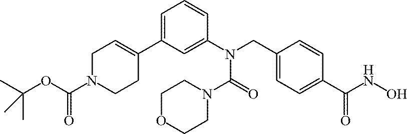 [SMILES: CC(C)(C)OC(=O)N1CC=C(c2cccc(N(Cc3ccc(C(=O)NO)cc3)C(=O)N3CCOCC3)c2)CC1] |
| 399 | 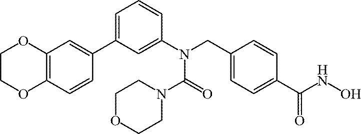 [SMILES: O=C(NO)c1ccc(CN(C(=O)N2CCOCC2)c2cccc(-c3ccc4c(c3)OCCO4)c2)cc1] | 413 | 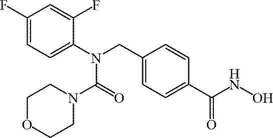 [SMILES: O=C(NO)c1ccc(CN(C(=O)N2CCOCC2)c2ccc(F)cc2F)cc1] |
| 400 | 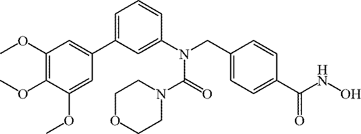 [SMILES: COc1cc(-c2cccc(N(Cc3ccc(C(=O)NO)cc3)C(=O)N3CCOCC3)c2)cc(OC)c1OC] | 414 | 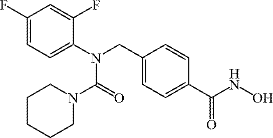 [SMILES: O=C(NO)c1ccc(CN(C(=O)N2CCCCC2)c2ccc(F)cc2F)cc1] |
| 415 | 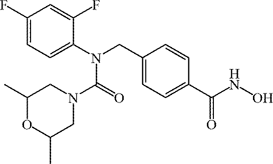 [SMILES: CC1CN(C(=O)N(Cc2ccc(C(=O)NO)cc2)c2ccc(F)cc2F)CC(C)O1] | 440 | 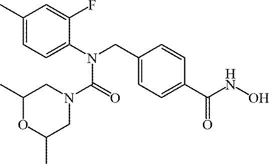 [SMILES: Cc1ccc(N(Cc2ccc(C(=O)NO)cc2)C(=O)N2CC(C)OC(C)C2)c(F)c1] |
| 416 | 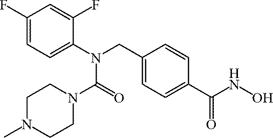 [SMILES: CN1CCN(C(=O)N(Cc2ccc(C(=O)NO)cc2)c2ccc(F)cc2F)CC1] | 441 | 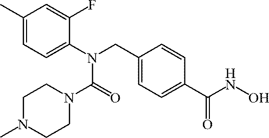 [SMILES: Cc1ccc(N(Cc2ccc(C(=O)NO)cc2)C(=O)N2CCN(C)CC2)c(F)c1] |
| 418 | 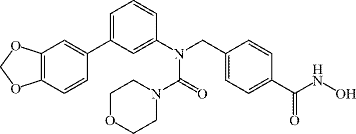 [SMILES: O=C(NO)c1ccc(CN(C(=O)N2CCOCC2)c2cccc(-c3ccc4c(c3)OCO4)c2)cc1] | 450 | 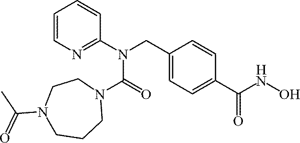 [SMILES: CC(=O)N1CCCN(C(=O)N(Cc2ccc(C(=O)NO)cc2)c2ccccn2)CC1] |
| 419 | 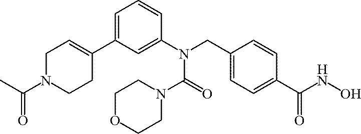 [SMILES: CC(=O)N1CC=C(c2cccc(N(Cc3ccc(C(=O)NO)cc3)C(=O)N3CCOCC3)c2)CC1] | 451 | 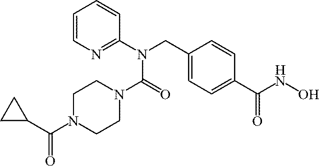 [SMILES: O=C(NO)c1ccc(CN(C(=O)N2CCN(C(=O)C3CC3)CC2)c2ccccn2)cc1] |
| 420 | 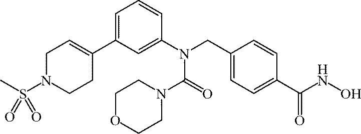 [SMILES: CS(=O)(=O)N1CC=C(c2cccc(N(Cc3ccc(C(=O)NO)cc3)C(=O)N3CCOCC3)c2)CC1] | 453 | 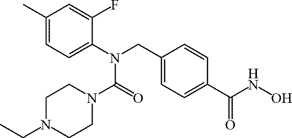 [SMILES: CCN1CCN(C(=O)N(Cc2ccc(C(=O)NO)cc2)c2ccc(C)cc2F)CC1] |
| 438 | 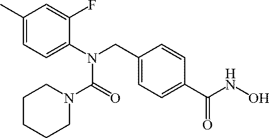 [SMILES: Cc1ccc(N(Cc2ccc(C(=O)NO)cc2)C(=O)N2CCCCC2)c(F)c1] | 454 | 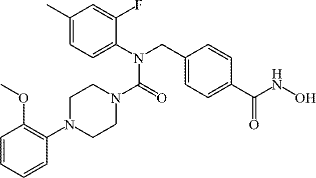 [SMILES: COc1ccccc1N1CCN(C(=O)N(Cc2ccc(C(=O)NO)cc2)c2ccc(C)cc2F)CC1] |
| 439 | 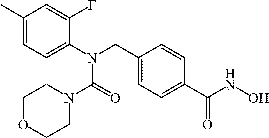 [SMILES: Cc1ccc(N(Cc2ccc(C(=O)NO)cc2)C(=O)N2CCOCC2)c(F)c1] | 455 | 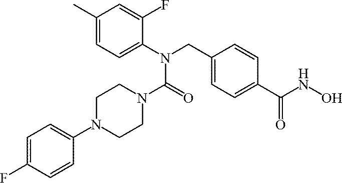 [SMILES: Cc1ccc(N(Cc2ccc(C(=O)NO)cc2)C(=O)N2CCN(c3ccc(F)cc3)CC2)c(F)c1] |
| 456 | 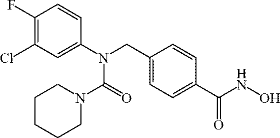 [SMILES: O=C(NO)c1ccc(CN(C(=O)N2CCCCC2)c2ccc(F)c(Cl)c2)cc1] | 463 | 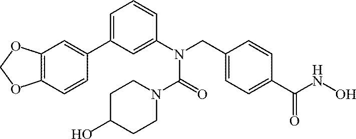 [SMILES: O=C(NO)c1ccc(CN(C(=O)N2CCC(O)CC2)c2cccc(-c3ccc4c(c3)OCO4)c2)cc1] |
| 457 | 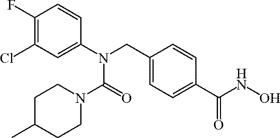 [SMILES: CC1CCN(C(=O)N(Cc2ccc(C(=O)NO)cc2)c2ccc(F)c(Cl)c2)CC1] | 464 | 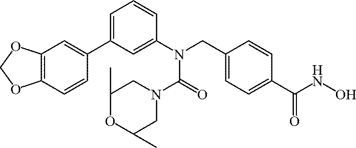 [SMILES: CC1CN(C(=O)N(Cc2ccc(C(=O)NO)cc2)c2cccc(-c3ccc4c(c3)OCO4)c2)CC(C)O1] |
| 458 | 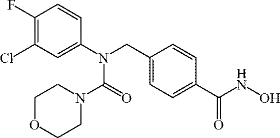 [SMILES: O=C(NO)c1ccc(CN(C(=O)N2CCOCC2)c2ccc(F)c(Cl)c2)cc1] | 465 | 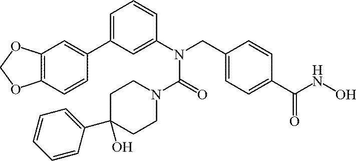 [SMILES: O=C(NO)c1ccc(CN(C(=O)N2CCC(O)(c3ccccc3)CC2)c2cccc(-c3ccc4c(c3)OCO4)c2)cc1] |
| 459 | 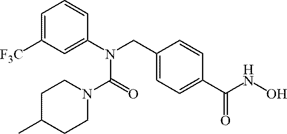 [SMILES: CC1CCN(C(=O)N(Cc2ccc(C(=O)NO)cc2)c2cccc(C(F)(F)F)c2)CC1] | 466 | 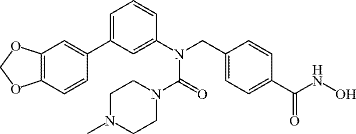 [SMILES: CN1CCN(C(=O)N(Cc2ccc(C(=O)NO)cc2)c2cccc(-c3ccc4c(c3)OCO4)c2)CC1] |
| 460 | 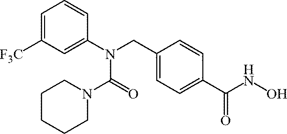 [SMILES: O=C(NO)c1ccc(CN(C(=O)N2CCCCC2)c2cccc(C(F)(F)F)c2)cc1] | 467 | 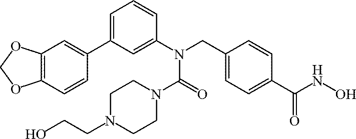 [SMILES: O=C(NO)c1ccc(CN(C(=O)N2CCN(CCO)CC2)c2cccc(-c3ccc4c(c3)OCO4)c2)cc1] |
| 461 | 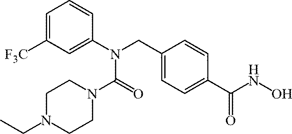 [SMILES: CCN1CCN(C(=O)N(Cc2ccc(C(=O)NO)cc2)c2cccc(C(F)(F)F)c2)CC1] | 468 | 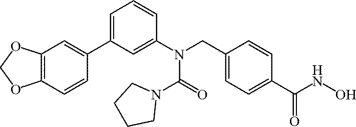 [SMILES: O=C(NO)c1ccc(CN(C(=O)N2CCCC2)c2cccc(-c3ccc4c(c3)OCO4)c2)cc1] |
| 462 | 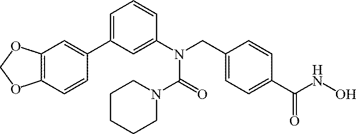 [SMILES: O=C(NO)c1ccc(CN(C(=O)N2CCCCC2)c2cccc(-c3ccc4c(c3)OCO4)c2)cc1] | 469 | 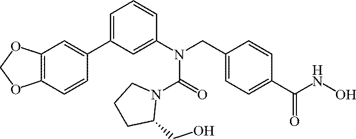 [SMILES: O=C(NO)c1ccc(CN(C(=O)N2CCC[C@H]2CO)c2cccc(-c3ccc4c(c3)OCO4)c2)cc1] |
| 470 | 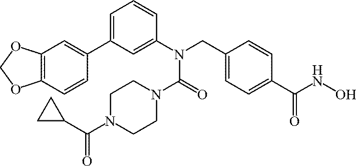 [SMILES: O=C(NO)c1ccc(CN(C(=O)N2CCN(C(=O)C3CC3)CC2)c2cccc(-c3ccc4c(c3)OCO4)c2)cc1] | 482 | 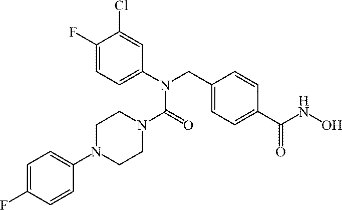 [SMILES: O=C(NO)c1ccc(CN(C(=O)N2CCN(c3ccc(F)cc3)CC2)c2ccc(F)c(Cl)c2)cc1] |
| 471 | 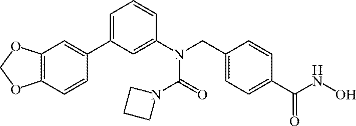 [SMILES: O=C(NO)c1ccc(CN(C(=O)N2CCC2)c2cccc(-c3ccc4c(c3)OCO4)c2)cc1] | 483 | 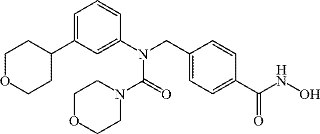 [SMILES: O=C(NO)c1ccc(CN(C(=O)N2CCOCC2)c2cccc(C3CCOCC3)c2)cc1] |
| 477 | 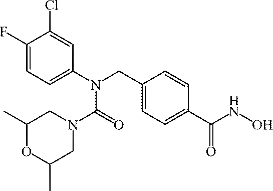 [SMILES: CC1CN(C(=O)N(Cc2ccc(C(=O)NO)cc2)c2ccc(F)c(Cl)c2)CC(C)O1] | 484 | 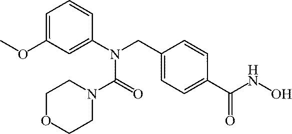 [SMILES: COc1cccc(N(Cc2ccc(C(=O)NO)cc2)C(=O)N2CCOCC2)c1] |
| 478 | 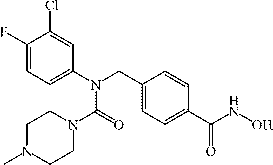 [SMILES: CN1CCN(C(=O)N(Cc2ccc(C(=O)NO)cc2)c2ccc(F)c(Cl)c2)CC1] | 485 | 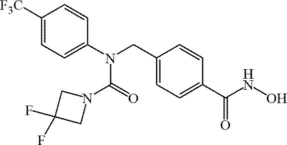 [SMILES: O=C(NO)c1ccc(CN(C(=O)N2CC(F)(F)C2)c2ccc(C(F)(F)F)cc2)cc1] |
| 479 | 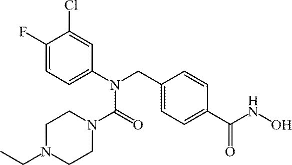 [SMILES: CCN1CCN(C(=O)N(Cc2ccc(C(=O)NO)cc2)c2ccc(F)c(Cl)c2)CC1] | 486 | 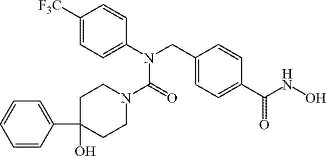 [SMILES: O=C(NO)c1ccc(CN(C(=O)N2CCC(O)(c3ccccc3)CC2)c2ccc(C(F)(F)F)cc2)cc1] |
| 480 | 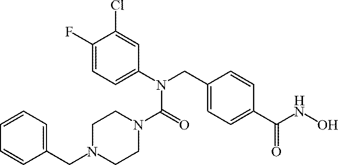 [SMILES: O=C(NO)c1ccc(CN(C(=O)N2CCN(Cc3ccccc3)CC2)c2ccc(F)c(Cl)c2)cc1] | 487 | 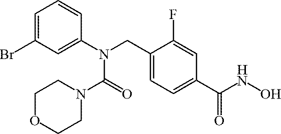 [SMILES: O=C(NO)c1ccc(CN(C(=O)N2CCOCC2)c2cccc(Br)c2)c(F)c1] |
| 481 | 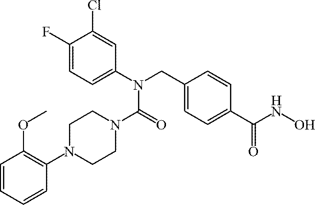 [SMILES: COc1ccccc1N1CCN(C(=O)N(Cc2ccc(C(=O)NO)cc2)c2ccc(F)c(Cl)c2)CC1] | 488 | 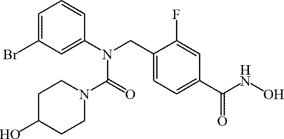 [SMILES: O=C(NO)c1ccc(CN(C(=O)N2CCC(O)CC2)c2cccc(Br)c2)c(F)c1] |
| 489 | 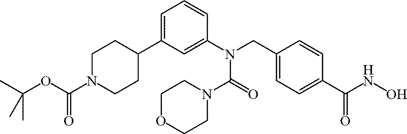 [SMILES: CC(C)(C)OC(=O)N1CCC(c2cccc(N(Cc3ccc(C(=O)NO)cc3)C(=O)N3CCOCC3)c2)CC1] | 496 | 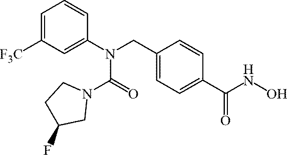 [SMILES: O=C(NO)c1ccc(CN(C(=O)N2CC[C@H](F)C2)c2cccc(C(F)(F)F)c2)cc1] |
| 490 | 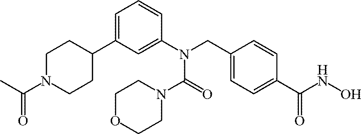 [SMILES: CC(=O)N1CCC(c2cccc(N(Cc3ccc(C(=O)NO)cc3)C(=O)N3CCOCC3)c2)CC1] | 497 | 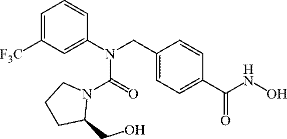 [SMILES: O=C(NO)c1ccc(CN(C(=O)N2CCC[C@@H]2CO)c2cccc(C(F)(F)F)c2)cc1] |
| 491 | 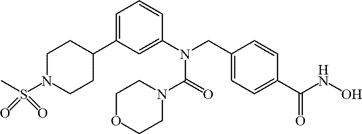 [SMILES: CS(=O)(=O)N1CCC(c2cccc(N(Cc3ccc(C(=O)NO)cc3)C(=O)N3CCOCC3)c2)CC1] | 498 | 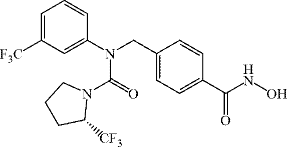 [SMILES: O=C(NO)c1ccc(CN(C(=O)N2CCC[C@H]2C(F)(F)F)c2cccc(C(F)(F)F)c2)cc1] |
| 492 | 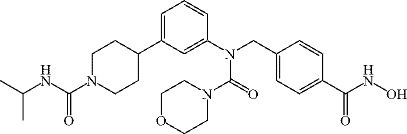 [SMILES: CC(C)NC(=O)N1CCC(c2cccc(N(Cc3ccc(C(=O)NO)cc3)C(=O)N3CCOCC3)c2)CC1] | 499 | 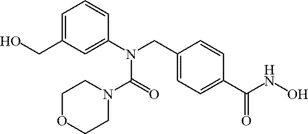 [SMILES: O=C(NO)c1ccc(CN(C(=O)N2CCOCC2)c2cccc(CO)c2)cc1] |
| 493 | 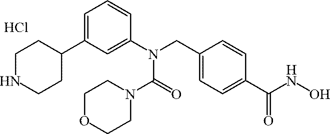 [SMILES: Cl ; O=C(NO)c1ccc(CN(C(=O)N2CCOCC2)c2cccc(C3CCNCC3)c2)cc1] | 500 | 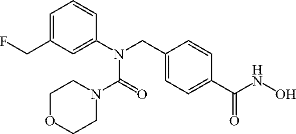 [SMILES: O=C(NO)c1ccc(CN(C(=O)N2CCOCC2)c2cccc(CF)c2)cc1] |
| 494 | 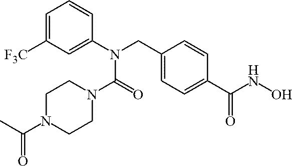 [SMILES: CC(=O)N1CCN(C(=O)N(Cc2ccc(C(=O)NO)cc2)c2cccc(C(F)(F)F)c2)CC1] | 511 | 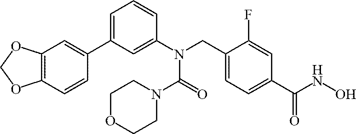 [SMILES: O=C(NO)c1ccc(CN(C(=O)N2CCOCC2)c2cccc(-c3ccc4c(c3)OCO4)c2)c(F)c1] |
| 495 | 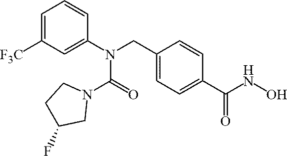 [SMILES: O=C(NO)c1ccc(CN(C(=O)N2CC[C@@H](F)C2)c2cccc(C(F)(F)F)c2)cc1] | 512 | 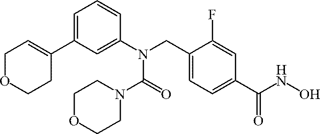 [SMILES: O=C(NO)c1ccc(CN(C(=O)N2CCOCC2)c2cccc(C3=CCOCC3)c2)c(F)c1] |
| 513 | 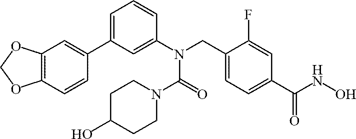 [SMILES: O=C(NO)c1ccc(CN(C(=O)N2CCC(O)CC2)c2cccc(-c3ccc4c(c3)OCO4)c2)c(F)c1] | 529 | 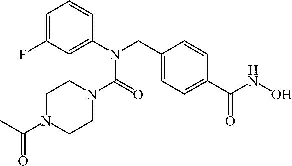 [SMILES: CC(=O)N1CCN(C(=O)N(Cc2ccc(C(=O)NO)cc2)c2cccc(F)c2)CC1] |
| 514 | 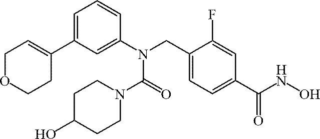 [SMILES: O=C(NO)c1ccc(CN(C(=O)N2CCC(O)CC2)c2cccc(C3=CCOCC3)c2)c(F)c1] | 530 | 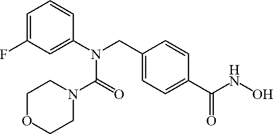 [SMILES: O=C(NO)c1ccc(CN(C(=O)N2CCOCC2)c2cccc(F)c2)cc1] |
| 517 | 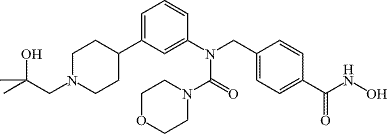 [SMILES: CC(C)(O)CN1CCC(c2cccc(N(Cc3ccc(C(=O)NO)cc3)C(=O)N3CCOCC3)c2)CC1] | 531 | 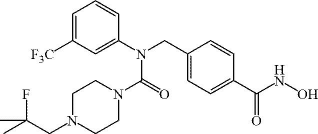 [SMILES: CC(C)(F)CN1CCN(C(=O)N(Cc2ccc(C(=O)NO)cc2)c2cccc(C(F)(F)F)c2)CC1] |
| 518 | 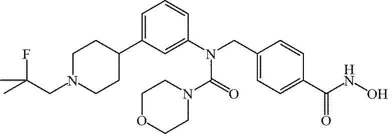 [SMILES: CC(C)(F)CN1CCC(c2cccc(N(Cc3ccc(C(=O)NO)cc3)C(=O)N3CCOCC3)c2)CC1] | 532 | 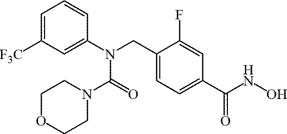 [SMILES: O=C(NO)c1ccc(CN(C(=O)N2CCOCC2)c2cccc(C(F)(F)F)c2)c(F)c1] |
| 520 | 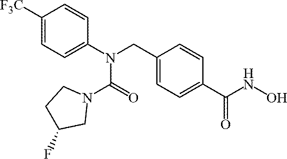 [SMILES: O=C(NO)c1ccc(CN(C(=O)N2CC[C@@H](F)C2)c2ccc(C(F)(F)F)cc2)cc1] | 533 | 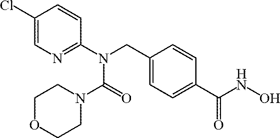 [SMILES: O=C(NO)c1ccc(CN(C(=O)N2CCOCC2)c2ccc(Cl)cn2)cc1] |
| 521 | 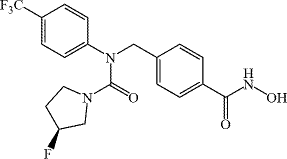 [SMILES: O=C(NO)c1ccc(CN(C(=O)N2CC[C@H](F)C2)c2ccc(C(F)(F)F)cc2)cc1] | 543 | 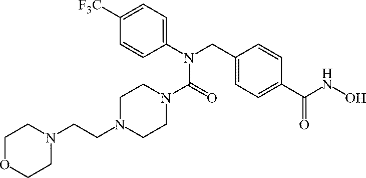 [SMILES: O=C(NO)c1ccc(CN(C(=O)N2CCN(CCN3CCOCC3)CC2)c2ccc(C(F)(F)F)cc2)cc1] |
| 522 | 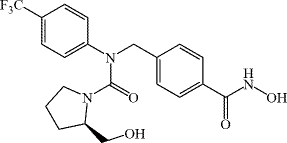 [SMILES: O=C(NO)c1ccc(CN(C(=O)N2CCC[C@@H]2CO)c2ccc(C(F)(F)F)cc2)cc1] | 544 | 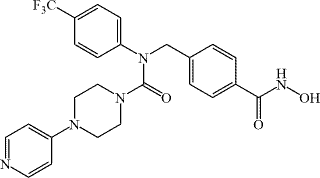 [SMILES: O=C(NO)c1ccc(CN(C(=O)N2CCN(c3ccncc3)CC2)c2ccc(C(F)(F)F)cc2)cc1] |
| 545 | 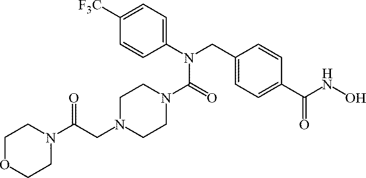 [SMILES: O=C(NO)c1ccc(CN(C(=O)N2CCN(CC(=O)N3CCOCC3)CC2)c2ccc(C(F)(F)F)cc2)cc1] | 716 | 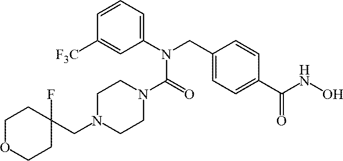 [SMILES: O=C(NO)c1ccc(CN(C(=O)N2CCN(CC3(F)CCOCC3)CC2)c2cccc(C(F)(F)F)c2)cc1] |
| 577 | 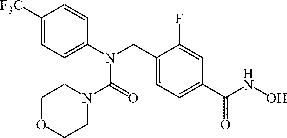 [SMILES: O=C(NO)c1ccc(CN(C(=O)N2CCOCC2)c2ccc(C(F)(F)F)cc2)c(F)c1] | 717 | 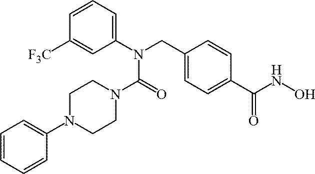 [SMILES: O=C(NO)c1ccc(CN(C(=O)N2CCN(c3ccccc3)CC2)c2cccc(C(F)(F)F)c2)cc1] |
| 578 | 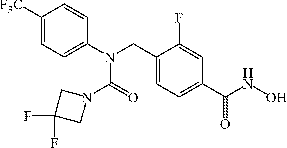 [SMILES: O=C(NO)c1ccc(CN(C(=O)N2CC(F)(F)C2)c2ccc(C(F)(F)F)cc2)c(F)c1] | 718 | 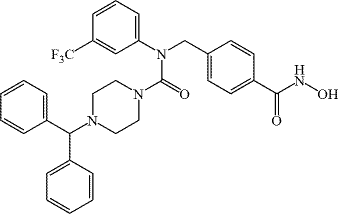 [SMILES: O=C(NO)c1ccc(CN(C(=O)N2CCN(C(c3ccccc3)c3ccccc3)CC2)c2cccc(C(F)(F)F)c2)cc1] |
| 580 | 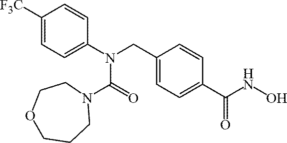 [SMILES: O=C(NO)c1ccc(CN(C(=O)N2CCCOCC2)c2ccc(C(F)(F)F)cc2)cc1] | 765 | 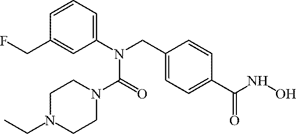 [SMILES: CCN1CCN(C(=O)N(Cc2ccc(C(=O)NO)cc2)c2cccc(CF)c2)CC1] |
| 651 | 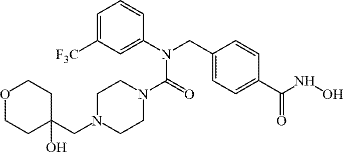 [SMILES: O=C(NO)c1ccc(CN(C(=O)N2CCN(CC3(O)CCOCC3)CC2)c2cccc(C(F)(F)F)c2)cc1] | 766 | 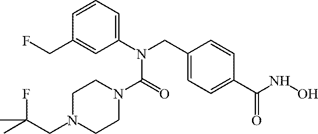 [SMILES: CC(C)(F)CN1CCN(C(=O)N(Cc2ccc(C(=O)NO)cc2)c2cccc(CF)c2)CC1] |
| 652 | 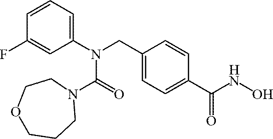 [SMILES: O=C(NO)c1ccc(CN(C(=O)N2CCCOCC2)c2cccc(F)c2)cc1] | 771 | 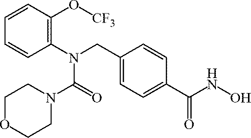 [SMILES: O=C(NO)c1ccc(CN(C(=O)N2CCOCC2)c2ccccc2OC(F)(F)F)cc1] |
| 683 | 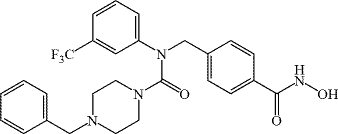 [SMILES: O=C(NO)c1ccc(CN(C(=O)N2CCN(Cc3ccccc3)CC2)c2cccc(C(F)(F)F)c2)cc1] | 772 | 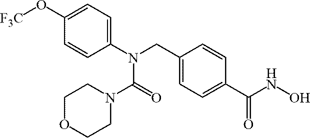 [SMILES: O=C(NO)c1ccc(CN(C(=O)N2CCOCC2)c2ccc(OC(F)(F)F)cc2)cc1] |
| 684 | 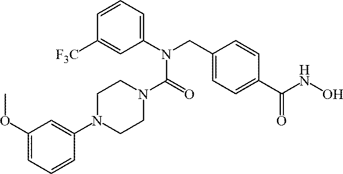 [SMILES: COc1cccc(N2CCN(C(=O)N(Cc3ccc(C(=O)NO)cc3)c3cccc(C(F)(F)F)c3)CC2)c1] | | |
| 773 | 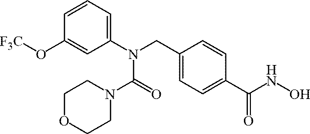 [SMILES: O=C(NO)c1ccc(CN(C(=O)N2CCOCC2)c2cccc(OC(F)(F)F)c2)cc1] | 801 | 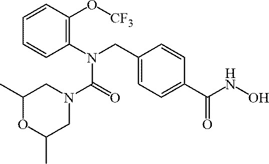 [SMILES: CC1CN(C(=O)N(Cc2ccc(C(=O)NO)cc2)c2ccccc2OC(F)(F)F)CC(C)O1] |
| 774 | 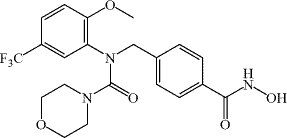 [SMILES: COc1ccc(C(F)(F)F)cc1N(Cc1ccc(C(=O)NO)cc1)C(=O)N1CCOCC1] | 802 | 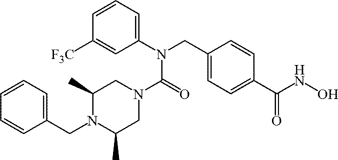 [SMILES: C[C@@H]1CN(C(=O)N(Cc2ccc(C(=O)NO)cc2)c2cccc(C(F)(F)F)c2)C[C@H](C)N1Cc1ccccc1] |
| 776 | 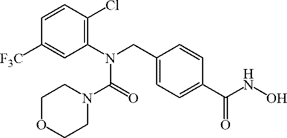 [SMILES: O=C(NO)c1ccc(CN(C(=O)N2CCOCC2)c2cc(C(F)(F)F)ccc2Cl)cc1] | 803 | 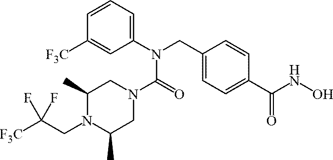 [SMILES: C[C@@H]1CN(C(=O)N(Cc2ccc(C(=O)NO)cc2)c2cccc(C(F)(F)F)c2)C[C@H](C)N1CC(F)(F)C(F)(F)F] |
| 778 | 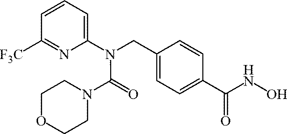 [SMILES: O=C(NO)c1ccc(CN(C(=O)N2CCOCC2)c2cccc(C(F)(F)F)n2)cc1] | 826 | 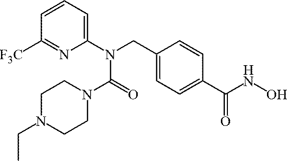 [SMILES: CCN1CCN(C(=O)N(Cc2ccc(C(=O)NO)cc2)c2cccc(C(F)(F)F)n2)CC1] |
| 791 | 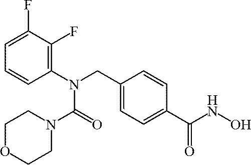 [SMILES: O=C(NO)c1ccc(CN(C(=O)N2CCOCC2)c2cccc(F)c2F)cc1] | 827 | 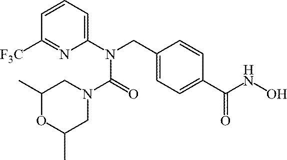 [SMILES: CC1CN(C(=O)N(Cc2ccc(C(=O)NO)cc2)c2cccc(C(F)(F)F)n2)CC(C)O1] |
| 797 | 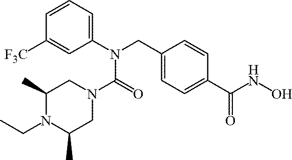 [SMILES: CCN1[C@H](C)CN(C(=O)N(Cc2ccc(C(=O)NO)cc2)c2cccc(C(F)(F)F)c2)C[C@@H]1C] | 828 | 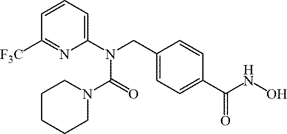 [SMILES: O=C(NO)c1ccc(CN(C(=O)N2CCCCC2)c2cccc(C(F)(F)F)n2)cc1] |
| 800 | 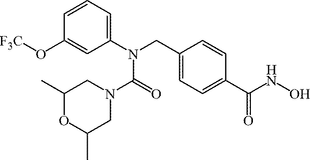 [SMILES: CC1CN(C(=O)N(Cc2ccc(C(=O)NO)cc2)c2cccc(OC(F)(F)F)c2)CC(C)O1] | 829 | 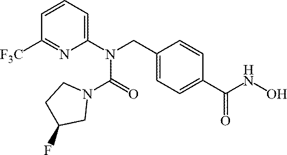 [SMILES: O=C(NO)c1ccc(CN(C(=O)N2CC[C@H](F)C2)c2cccc(C(F)(F)F)n2)cc1] |

.

5. The pharmaceutical composition according to claim 1, wherein the compound represented by the above chemical formula I is a compound described in a following table:

| Compou nd | Structure | Compo und | Structure |
|---|---|---|---|
| 374 | 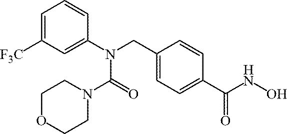 [SMILES: O=C(NO)c1ccc(CN(C(=O)N2CCOCC2)c2cccc(C(F)(F)F)c2)cc1] | 413 | 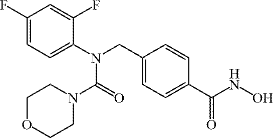 [SMILES: O=C(NO)c1ccc(CN(C(=O)N2CCOCC2)c2ccc(F)cc2F)cc1] |
| 458 | 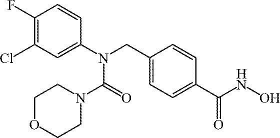 [SMILES: O=C(NO)c1ccc(CN(C(=O)N2CCOCC2)c2ccc(F)c(Cl)c2)cc1] | 484 | 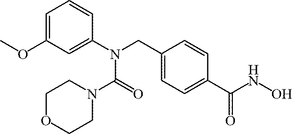 [SMILES: COc1cccc(N(Cc2ccc(C(=O)NO)cc2)C(=O)N2CCOCC2)c1] |
| 530 | 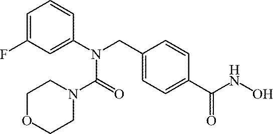 [SMILES: O=C(NO)c1ccc(CN(C(=O)N2CCOCC2)c2cccc(F)c2)cc1] | 652 | 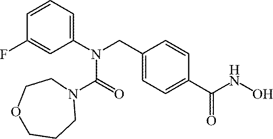 [SMILES: O=C(NO)c1ccc(CN(C(=O)N2CCCOCC2)c2cccc(F)c2)cc1] |

.

6. The pharmaceutical composition according to claim 1, wherein said pharmaceutical composition is orally administered.

7. A method for preventing or treating chronic obstructive pulmonary disease, the method comprising administering a therapeutically effective amount of a compound represented by a chemical formula I according to claim 1, optical isomers thereof or pharmaceutically acceptable salts thereof into an individual.

8. A use of a compound represented by a chemical formula I according to claim 1, optical isomers thereof or pharmaceutically acceptable salts thereof for preventing or treating chronic obstructive pulmonary disease.

9. A use of a compound represented by a chemical formula I according to claim 1, optical isomers thereof or pharmaceutically acceptable salts thereof in preparation of a medicament for treating chronic obstructive pulmonary disease.


SEQUENCE LISTING (8 sequences)

SEQ ID NO: 1 - DNA, 21 bp, Artificial Sequence
  Primer(F)_mIL-6
ctgcaagagacttccatccag

SEQ ID NO: 2 - DNA, 23 bp, Artificial Sequence
  Primer(R)_mIL-6
agtggtatagacaggtctgttgg

SEQ ID NO: 3 - DNA, 21 bp, Artificial Sequence
  Primer(F)_mIFN-gamma
atgaacgctacacactgcatc

SEQ ID NO: 4 - DNA, 21 bp, Artificial Sequence
  Primer(R)_mIFN-gamma
ccatccttttgccagttcctc

SEQ ID NO: 5 - DNA, 23 bp, Artificial Sequence
  Primer(F)_mMCP-1
ttaaaaacctggatcggaaccaa

SEQ ID NO: 6 - DNA, 23 bp, Artificial Sequence
  Primer(R)_mMCP-1
gcattagcttcagatttacgggt

SEQ ID NO: 7 - DNA, 20 bp, Artificial Sequence
  Primer(F)_mTNF-alpha
ctgaacttcggggtgatcgg

SEQ ID NO: 8 - DNA, 23 bp, Artificial Sequence
  Primer(R)_mTNF-alpha
ggcttgtcactcgaattttgaga